(19) United States
(12) Patent Application Publication
(10) Pub. No.: US 2002/0137717 A1
(43) Pub. Date: Sep. 26, 2002

(54) Use of chimeric mutational vectors to change endogenous nucleotide sequences in solid tissues

(76) Inventors: Richard J. Bartlett, Weston, FL (US); Thomas A. Rando, Stanford, CA (US)

Correspondence Address: PILLSBURY WINTHROP, LLP, P.O. BOX 10500, MCLEAN, VA, 22102, US

(21) Appl. No.: 10/077,894

(22) Filed: Feb. 20, 2002

Related U.S. Application Data
(63) Continuation of application No. 09/576,081, filed on May 20, 2000, now abandoned.
(60) Provisional application No. 60/135,139, filed on May 21, 1999.
(60) Provisional application No. 60/174,388, filed on Jan. 3, 2000.

(51) Int. Cl. A61K 48/00

(52) U.S. Cl. 514/44

(57) ABSTRACT

This invention relates to the field of muscular dystrophy and methods for its treatment in humans. This invention also concerns art-recognized animal models of Duchenne muscular dystrophy in dogs (GRMD) and mice (mdx). Another aspect concerns chimeric mutational vectors capable of inducing reversion of genetic mutations (i.e., gene repair) causing genetic disease by direct injection into affected tissue. Thus, more generally, the invention envisions direct injection of chimeric mutational vectors into affected tissues to effect gene repair therein.

### CROSS-REFERENCES TO RELATED APPLICATIONS

This is a continuation application which claims priority benefit to U.S. patent application Ser. No. 09/576,081, filed May 20, 2000, which claims priority benefit to provisional U.S. Appln. No. 60/135,139, filed May 21, 1999, and provisional U.S. Appln. No. 60/174,388, filed Jan. 5, 2000, all of which are incorporated by reference herein.

### FIELD OF THE INVENTION

The invention concerns methods of treating genetic diseases or other pathologic conditions by making one or more specific changes in endogenous nucleotide sequences of solid tissues. These specific changes are mediated by oligonucleobases called chimeric mutational vectors (CMV). The CMV can be administered directly to the subject in vivo; in particular, the CMV can be injected into a solid tissue in which expression of the mutated gene occurs. Such gene repair can reverse the disease or other pathologic condition caused by the mutation or, alternatively, can introduce a second change that compensates for the disease or condition causing mutation.

### BACKGROUND OF THE INVENTION

The inclusion of a reference in this section is not to be understood as an admission that its teachings were publicly available prior to our invention of the subject matter disclosed herein or that they resulted from someone other than the inventors.

Chimeric Mutational Vector (CMV)

An oligonucleobase, which has complementary segments of deoxyribonucleotides and ribonucleotides, and contained a sequence homologous to a fragment of the bacteriophage M13mp19, has been described (Kmiec et al., Molecular and Cellular Biology 14:7163-7172, 1994). The oligonucleobase had a single contiguous segment of ribonucleotides. It is a substrate for the REC2 homologous pairing enzyme from Ustilago maydis. Thus, this enzyme and the DNA mismatch repair machinery were suggested to be involved in gene repair.

Patent publication WO 95/15972, published June 15, 1995, and corresponding U.S. application Ser. No. 08/353,657, filed Dec. 9, 1994, now U.S. Pat. No. 5,565,350 (the '350 patent) described chimeraplasts to genetically change eukaryotic cells. Examples with a Ustilago maydis gene and the murine ras gene were reported. The latter example was designed to introduce a transforming mutation into the ras gene so that the successful mutation of the ras gene in murine NIH 3T3 cells would cause the growth of a colony of cells. The maximum rate of such transformation of cultured cells was less than 0.1%, i.e., less than 100 transformants per 10⁶ cells exposed to the CMV had a phenotype indicative of ras mutation. In the Ustilago maydis system, the rate of introduction of the genetic change was about 600 per 10⁶ cells. A chimeraplast was also designed to introduce a mutation into the human bcl-2 gene (Kmiec, Seminars in Oncology 23:188-193, 1996).

A chimeraplast was also designed to repair a mutation in codon 12 of K-ras (Kmiec, Advanced Drug Delivery Reviews 17:333-340, 1995). The chimeraplast was introduced into Capan 2, a cell line derived from a human pancreatic adenocarcinoma, using LIPOFECTIN cationic lipid. Twenty-four hours after the chimeraplasts were introduced, cells were harvested and genomic DNA was extracted. A fragment containing codon 12 of K-ras was amplified by PCR and the rate of conversion estimated by hybridization with allele-specific probes. The rate of repair was reported to be approximately 18%.

A chimeraplast has been designed to repair a mutation in the gene encoding liver/bone/kidney type alkaline phosphatase (Yoon et al., Proceedings of the National Academy of Sciences USA 93:2071-2076, 1996). The alkaline phosphatase gene was transiently introduced into CHO cells by a plasmid. Six hours later the chimeraplasts were introduced. The plasmid was recovered at 24 hours after introduction of the chimeraplast and analyzed. The results showed that approximately 30 to 38% of the alkaline phosphatase genes were repaired by the chimeraplast.

U.S. application Ser. No. 08/640,517, filed May 1, 1996 and published as WO 97/41141, and Cole-Strauss et al., Science 273:1386 1389, 1996, disclose chimeraplasts that are used in the treatment of genetic diseases of hematopoietic cells, e.g., sickle cell disease, thalassemia, and Gaucher disease. U.S. application Ser. No. 08/664,487, filed Jun. 17, 1996 and published as WO 97/04871, describes chimeraplast having non-natural nucleotides for use in specific, site-directed mutagenesis. The chimeraplasts described in the applications and publications of Kmiec and his colleagues contain a central segment of DNA:DNA homoduplex and flanking segments of RNA:DNA heteroduplex or 2′-O-Me-RNA:DNA heteroduplex. Kren et al., Hepatology 25:1462-1468, 1997, report the successful use of a CMV in non-replicating primary hepatocytes.

U.S. Appln. No. 60/054,837, filed Aug. 5, 1997, U.S. application Ser. No. 09/108,006, filed Jun. 30, 1998, and U.S. Appln. No. 60/064,996, filed Nov. 5, 1997, concern the use of chimeraplasts in non-replicating cells and compositions of CMV and macromolecular carriers, including macromolecular carriers that have ligands for clathrin-coated pit receptors.

Introduction of DNA into Muscle Cells

There are several references that report the introduction and expression of plasmid DNA encoding the dystrophin protein into skeletal muscle (Acsadi et al., Nature 352:815-818, 1991; Danko et al., Human Molecular Genetics 2:2055-2061, 1993; Bartlett et al., Cell Transplantation 5:411-419, 1996; Wells et al., FEBS Letters 332:179-182, 1993; Fritz et al., Pediatric Research. 37:693-700, 1995; Wolff et al., Human Molecular Genetics 1:363-369, 1992; Inui et al., Brain & Development 18:357-361, 1996). A general method of introducing DNA into a muscle cell for the purpose of inducing an immune response in a host is disclosed in U.S. Pat. Nos. 5,589,466 and 5,580,859. The expression of an exogenous dystrophin gene is an example in these patents.

Experiments directed at determining a ligand that can be used to introduce large DNA fragments into the myofibers of DMD patients have been reported (Feero et al., Gene Therapy 4:664-674, 1997). The use of liposomes to deliver DNA to myofibers for expression without the use of a targeting ligand has also been described (Templeton et al., Nature Biotechnology 15:647-652, 1997).

Molecular Biology of Muscular Dystrophy

The muscular dystrophies comprise a genetically and clinically diverse set of diseases characterized by abnormalities of the skeletal muscle (reviewed by Straub et al., Current Opinion in Neurology 10:168-175, 1997). The muscular dystrophies can be classified by the mode of inheritance, i.e., autosomal dominant, autosomal recessive, and X-linked, and each type further divided according to the chromosomal locus and even the effected gene, if known.

The most common muscular dystrophy is X-linked with the dystrophin gene effected. The dystrophin gene occupies 2,300 kb or about 1.5% of the X-chromosome. Its mature transcript is 14 kb and encodes a protein of 3685 amino acids having a molecular weight of 427 kd. The gene contains 79 exons. The dystrophin gene is extraordinarily large; it is about half the size of an E. coli genome. There is no clear explanation for its size. See Worton & Brooks, The Metabolic and Molecular Basis of Inherited Disease 7th Ed. Chapter 140 (McGraw Hill, New York, 1995).

The dystrophin protein contains an N-terminal binding region, that binds to intracellular filamentous actin (which is not the actin of the contractile apparatus), a C-terminal binding domain that binds to a transmembranous glycoprotein complex which in turn binds to laminin, and a connective region. Under physiologic conditions, dystrophin exists as a homodimer and connects the actin filaments with the glycoprotein complex as well as linking each.

Although there are multiple mutations of dystrophin that result in muscular dystrophy, the mutations can be classified into types. The milder form, termed Becker Muscular Dystrophy (BMD), is associated with genomic deletions or mRNA processing errors that do not alter the reading frame of the mature mRNA and, hence result in a mutant protein that contains intact N-terminal and C-terminal binding domains. In the more severe form, termed Duchenne Muscular Dystrophy (DMD), the dystrophin protein lacks a C-terminal binding domain and is usually unstable. DMD typically results from point mutations that introduce in-frame termination codons or from insertion or deletion mutations that result in a frame-shift. See, generally, Koenig et al., American Journal of Human Genetics 45:498-506,1989; Prior et al., Human Mutation 5:263-268, 1995; Koenig et al., Cell 50:509-517, 1987; Baumbach et al., Neurology 39:465-474, 1989.

The relationship between the pathophysiology of DMD and BMD and the physiologic function of dystrophin is complex. Dystrophin is not required to transmit the force of the contractile apparatus to the tendonous connections of the muscle. Rather, the defective muscles undergo an ongoing series of focal necrosis of the myofibers, which ultimately exceed the repair capacity of the muscle. The end stage disease is characterized by fibrosis between myofibers, atrophy, and weakness.

Dystrophin Replacement Gene Therapy

Several groups have attempted to treat DMD by introducing genes encoding dystrophin into the myofibers of affected individuals. A variety of methods have been employed and can be classified into three groups: in situ replacement gene therapy; ex vivo replacement gene therapy using autologous myoblasts, which are then reimplanted; and allogenic transplantation of wild-type myoblasts.

Examples of the first type include the aforementioned transfections of differentiated myofibers using DNA and non-biologic carriers. This form of therapy has been of limited value because of the low efficiency of transfection. The use of adenovirus based vectors to increase efficiency has been reported. See, generally, Vincent et al., Nature Genetics 5:130-134, 1993; Ragot et al., Gene Therapy 1 Suppl 1:S53-S54, 1994; Acsadi et al., Human Gene Therapy 7:129-140, 1996; Deconinck et al., Proceedings of the National Academy of Sciences USA 93:3570-3574, 1996; Clemens et al., Gene Therapy 3:965-972, 1996; Haecker et al., Human Gene Therapy 7:1907-1914, 1996; Chen et al., Proceedings of the National Academy of Sciences USA 94:1645-1650, 1997; Yang et al., Journal of Virology 69:2004-2015, 1995; Haecker et al., Human Gene Therapy 7:1907-1914, 1996. Although efficiencies as high as 50% have been reported in experimental animal systems (Ragot et al., Nature 361:647-650, 1993), adenovirus-based therapies have likewise been of limited value to date because the expression of dystrophin has been transient and there is an inunune response to the adenovirus vector that limits the possibilities of repeated therapy. Although such gene therapy has not proved to be a practical clinical modality, it has been useful to demonstrate that the expression of a wild-type dystrophin in an DMD model system results in amelioration of the disease (Danko et al., Human Molecular Genetics 2, 2055-2061, 1993).

Techniques for the culture of myoblasts from normal individuals have been reported (U.S. Pat. No. 5,538,722). Dystrophin has been transferred into cultured myoblasts (Dunckley et al., FEBS Letters 296:128-134, 1992) but this approach has not been pursued because a secondary effect of DMD is a decline in the numbers of myoblasts that can be recovered in culture (Webster & Blau, Somatic Cell & Molecular Genetics 16:557-565, 1990).

Successful engraftment of allogenic cultured myoblasts has been reported (U.S. Pat. No. 5,130,141; Law et al., Cellular Transplantation 1:235-244, 1992). Other studies, however, have failed to confirm the clinical benefit of allogenic myoblast grafts under controlled conditions (Gussoni et al., Nature 356:435-438, 1992; Karpati et al., Annals of Neurology 34:8-17, 1992). There is consequently a need for a therapy that results in the long-term expression of functional dystrophin in muscle fibers affected by muscular dystrophy. Ideally, the therapy should be applicable to all solid tissues whether or not they are highly vascularized.

A further limitation of both myoblast engraftment and non-viral gene therapy is a requirement for local delivery, such that multiple injections are required to treat even a single large muscle and obtain permanent effects (e.g., gene repair). Reports to the contrary with regard to myoblast engraftment notwithstanding (e.g., Hughes & Blau, Nature 345:350-353, 1990; Neumeyer et al., Neurology 42:2258-2262, 1992), more recent studies have not confirmed that transvascular engraftment into muscle fibers occurs to any practical extent.

Two well-characterized animal models exist for Duchenne muscular dystrophy, the mdx mouse (Bulfield et al., Proceedings of the National Academy of Sciences USA 81:1189-1192, 1984; Sicinski et al., Science 244:1578-1579, 1989) and the golden retriever dog (Kornegay et al., Muscle and Nerve 11:1056-1064, 1988; Sharp et al., Genomics 13:115-121, 1992). In both cases, a point mutation has been identified as causing disease: the mouse having a nonsense mutation in exon 23 and the dog having a splice acceptor site mutation in intron 6 causing a frame-shift due to complete deletion of exon 7 from the mature canine dystrophin mRNA (Wilton et al., Muscle and Nerve 20:728-734, 1997; Wilton et al., Neuromuscular Disorders 7:329-335, 1997; Schatzberg et al., Muscle and Nerve 21:991-998, 1998). Alternate splicing mechanisms, which restore the dystrophin reading via removal of mutation containing out-of-frame exons, have been suggested to play a causal role for the presence of dystrophin positive staining “revertant fibers” in both models, although no evidence of true reversion of these point mutations at the genomic level have been reported. A considerable amount of effort has gone into the study of gene therapy in the mdx model using direct DNA injection (Acsadi et al., Nature 352:815-818, 1991) viral vectors (Danko et al., Human Molecular Genetics 2:2055-2061, 1993; Wells et al., FEBS Letters 332:179-182, 1992) and myoblast transplantation (Fritz et al., Pediatric Research 37:693-700, 1995; Inui et al., Brain & Development 18:357-361, 1996) with modest levels of short-term success due to limitations of transfection targeting and efficiency, and either acute or chronic immune responses directed against cells which express the therapeutic gene product (Kinoshita et al., Acta Neuropathologica 91:489-493, 1996; Kinoshita et al., Neuromuscular Disorders 6:187-193, 1996; Yang et al., Journal of Virology 70:7209-7212, 1996; Yang et al., Gene Therapy 3:137-144, 1996; Worgall et al., Human Gene Therapy 8:37-44, 1997). Recent studies have suggested that myoblast transplantation therapy of Duchenne muscular dystrophy is also ineffective (Partridge et al., Nature Medicine 4:1208-1209, 1998; Mendell et al., New England Journal of Medicine 333:832-838, 1995). Long-term correction of dystrophin deficiency requires a permanent effect such as gene repair which will provide stable expression of dystrophin without the problems associated with therapies such as delivery of expression vectors, viruses, and cell implantation.

Recently, a novel chimeric RNA and DNA oligonucleotide (i.e., a type of chimeraplast) was used to correct the sickle-cell globin allele in a lymphoblast cell line (Cole-Strauss et al., Science 273:1386-1389, 1996). This technique, termed chimeraplasty, is believed to rely on regions of sequence homology (i.e., mutator regions) designed into the chimeraplast that brackets the site of the chromosomal mutation and directs the host cell DNA mismatch repair mechanism to correct the endogenous sequence to that designated within the mutator region (Ye et al., Molecular Medicine Today 4:431-437, 1998). In the sickle cell study, this resulted in the correction to the wild-type nucleotide sequence of 20% of the chromosomes bearing the sickle-cell globin mutation.

A critical issue in the field of gene therapy is reliable and safe introduction of nucleic acid into the subject's cells. Introduction of large, highly charged molecules (e.g., expression vectors used in gene therapy) has proved challenging, and current protocols have been very limited and generally laborious. Thus, we show that chimeric mutational vectors and direct injection into solid tissue affected by a genetic mutation improves the efficiency of gene repair in well-characterized animal models of a human genetic disease. In particular, products and processes effective for introducing the chimeric mutational vector into cells of skeletal muscle (e.g., myoblasts, myocytes, myotubes, myofibers), and thereby correct dystrophin mutations therein, are provided. Similar products and processes are envisioned for other inherited and acquired genetic mutations. Other advantages of the invention beside those noted above will be appreciated by a person skilled in the art from the description below.

### SUMMARY OF THE INVENTION

A composition is provided that includes at least one chimeric mutational vector (CMV). Methods of making and using such compositions, which are used to change an endogenous nucleotide sequence of an affected cell in solid tissue and thereby correct a genetic mutation that causes a disease or other pathologic condition, are also provided.

Introducing at least one chimeric mutational vector (CMV) can mediate one or more sequence-specific changes in the endogenous sequence of at least some cells of the solid tissue. Applications of this invention are not limited to repair of a gene's coding sequences because non-coding and other chromosomal sequences could also be changed. For example, point mutations (e.g., nonsense or missense changes) and frame-shift mutations (e.g., insertions or deletions) in the coding region of a gene could be repaired, as well as genetic mutations in transcriptional regulatory regions (e.g., promoter, silencer, enhancer), initiation and termination sites for transcription or translation, or splice donors/acceptors.

We illustrate the operation of the invention by correction of dystrophin mutations in skeletal muscle. But more generally, any disease or other pathologic condition could be treated if the genetic basis was known: e.g., factor VIII and factor IX of liver for hemophilia A and B, respectively; UDP-glucuronosyltransferase of liver for Crigler-Najjar syndrome; expression of tyrosine hydroxylase or other enzymes involved in L-dopamine biosynthesis could be increased in the substantia nigra to treat Parkinson's disease. Other mutated genes in liver which could be changed by this invention are also known to cause familial hypercholesterolemia, mucopoly-saccharidosis, familial amyloidosis, phenylketonuria, maple syrup urine disease, hemochroma-tosis, α1-antitrypsin deficiency, Wilson's disease, and omithine transcarbamylase deficiency. Moreover, beneficial mutations could be made in a “normal” gene to prevent disease: e.g., APOB 100 may could be truncated or APO Al may be altered to the Milano allele to increase serum high-density lipoproteins (HDL), and thereby reduce the circulating amount of low-density lipoproteins (LDL). See Scriver et al. (eds.), Metabolic Basis of Inherited Disease, McGraw-Hill (New York, N.Y., 1993) and Online Mendelian Inheritance in Man, OMIM database, Center for Medical Genetics, Johns Hopkins University (Baltimore, Md.) and National Center for Biotechnology Information, National Library of Medicine (Bethesda, Md.) at http://www.ncbi.nlm.nih.gov/Omim/ for further information on human diseases and pathologic conditions for which genes and mutations have been identified. Mutations in oncogenes and tumor suppressor genes could also be repaired to treat neoplastic disease (e.g., cell cycle regulatory genes, DNA repair gene). For example, it might be possible to treat cancers of the muscle (e.g., sarcoma), liver (i.e., hepatoma), skin (e.g., melanoma), or brain (e.g., glioblastoma).

Gene repair is a process by which a specific alteration is introduced into an existing gene of a cell of the subject suffering from a disease. Gene repair differs from gene therapy in that gene therapy introduces an exogenous DNA fragment into the genome of a cell that is then expressed as the protein encoded by the introduced fragment. Gene repair, however, directs the DNA repair process of the subject cell to introduce the desired, specific alteration into the genome of the host cell. CMV does not need to be transcribed into an RNA transcript and does not have to encode a functional protein. This invention is based on the discoveries that CMV can be efficiently introduced into cells of solid tissues and that their nuclei are able to effect gene repair. Thus, delivery of a CMV into a cell is able to mediate a specific sequence change at high efficiency in vivo, and without the need for in vitro tissue culture or selection.

The sequence-specific genetic alteration can be made using a CMV as in “naked” form or in a delivery vehicle. Transfection agents such as, for example, lipids, viral particle, salt and polymeric precipitants, etc., may or may not be used to aid the introduction of the CMV into at least some cells of the solid tissue. Furthermore, the CMV may or may not be complexed with a macromolecular carrier to which is attached a specific ligand, e.g., a glucosyl moiety. The ligand may also be selected to bind to a cell-surface receptor that is internalized into the cell through clathrin-coated pits into endosomes. Alternatively, the CMV may be linked directly to the ligand without employing an intermediate macromolecular carrier. Targeted delivery of the CMV may also be achieved by using a ligand for a cellular receptor found specifically on the target tissue which is endocytosed. Other tissues which may be targeted include nervous tissues (e.g., brain, eye, central and peripheral nerves, glia); hematopoietic tissues (e.g., bone marrow, liver); reproductive tissues and glands (e.g., breast, adrenal gland, pituitary gland, thyroid gland); connective tissues, smooth muscle, striated muscle (e.g., skeletal, heart), and skin; and other solid tissues. Another optional additive is one that can be used to indicate the injection track of the composition in a treated solid tissue.

In alternative embodiments, the invention concerns the ex vivo use of gene repair to correct genetic mutations in cultured autologous cells of the solid tissue, which can then be engrafted into a subject. Furthermore, in utero use of gene repair may correct mutations prior to development of symptoms and when the number of cells in the solid tissue is reduced. Expansion of cells whose genetic mutations have been corrected because of a selective growth advantage conferred by the functional gene and/or by induction of regeneration (e.g., barium chloride for muscle) can be used to increase the proportion of cells in the solid tissue that have undergone gene repair.

Our invention is described below and its advantages over the prior art are illustrated by way of those particular embodiments and certain technical features.

### BRIEF DESCRIPTION OF THE DRAWINGS

FIG. 1 is a schematic of a chimeric mutational vector (CMV).

FIG. 2 shows the normal human nucleotide sequence (SEQ ID NO:1), the normal canine nucleotide sequence (SEQ ID NO:2), the GRMD mutant nucleotide sequence (SEQ ID NO:3), and the nucleobase sequence of the CMV used for repair of the GRMD mutation (SEQ ID NO:4). The CMV sequence has a two-base mismatch as compared to the canine sequence designed to help distinguish both mutant and wild-type sequences from the repaired sequence.

FIG. 3 shows a timeline for injections (dark vertical arrow and horizontal line for left limb treatment and cross-hatched vertical arrow and horizontal line for right limb treatment) and biopsies. The elapsed time until necroscopy was 48 weeks for the left limb and 39 weeks for the right limb.

FIG. 4 shows the locations of primers and mutations in the canine dystrophin gene.

FIG. 5 shows a normal nucleotide sequence (SEQ ID NO:5) and the mdx mutation (SEQ ID NO:6) in panel A, the design of chimeric mutational vector MDX1 designed to repair the mdx mutation in a murine dystrophin gene (SEQ ID NO:7) in panel B, and a putative mechanism for gene repair to produce a corrected mdx allele (SEQ ID NO:8) in panel C.

### DETAILED DESCRIPTION OF THE INVENTION

Multiple lines of evidence confirm that direct in vivo injection into dystrophic skeletal muscle of an appropriately designed and synthesized chimeric oligonucleobase (i.e., a chimeric mutational vector or CMV) results in reversion of the genetic mutation causing GRMD in dogs and the mdx mutation in mice. It is envisioned that such CMV-mediated gene repair can also be accomplished in humans having Duchenne and Becker muscular dystrophy. We have also surprisingly found that use of a lipid carrier vehicle to introduce the CMV into cells with a dystrophin mutation was required in dogs for sustained expression of corrected dystrophin transcripts, while successful gene repair of a point mutation in mice was not so limited.

In accordance with these teachings, those skilled in the art will appreciate that the invention can be used to treat muscluar dystrophies caused by mutations in 30 genes other than dystrophin. For example, the invention can also be used to correct mutations in Emery-Dreifuss muscular dystrophy caused by mutations in emerin, an X-linked gene, and recessive limb-girdle muscular dystrophy caused by mutations in the sarcoglycoan genes, which are encoded on autosomes.

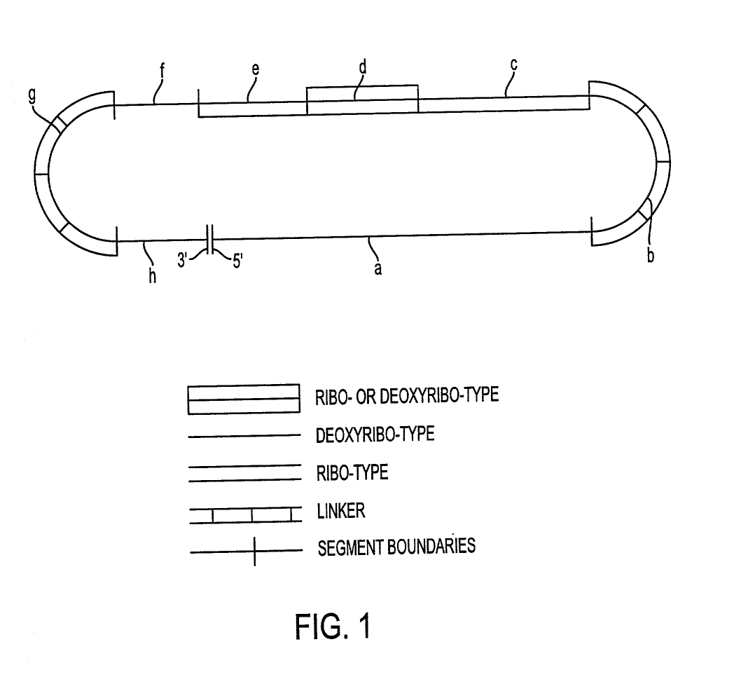

FIG. 1 shows a diagram of a CMV according to one embodiment of the invention. Segments “a” and “c-e” are target gene specific segments of the CMV. The sequences of segment “a” and “c-e” are complements of each other. The sequence of segments “f” and “h” are also complements of each other but are unrelated to the specific target gene and are selected merely to ensure the stability of hybridization in order to protect the 3′ and 5′ ends. Additional protection of the 3′ and 5′ ends can be accomplished by making the 5′ and 3′ most internucleobase bonds a phosphorothioate, phosphonate or any other nuclease-resistant bond. The sequence of segments “f” and “h” can be 5′-GCGCG-3′ or permutations thereof. Segments “g” and “b” can be any linker that covalently connects the two strands, e.g., four unpaired nucleotides or an alkoxy oligomer such as polyethylene glycol. When segments “g” and “b” are composed of other than nucleobases, then segments “a”, “c-f” and “g” are each an oligonucleobase chain. The ribo-type nucleobase segments are segments “c” and “e,” which form hybrid-duplexes by Watson-Crick base pairing to the complementary portions of segment “a.” The segment “a” can have the sequence of either the coding or non-coding strand of the gene.

The sequence of the CMV useful to treat a particular subject depends upon the location and type of the mutation of the subject. Mutations consisting of the replacement of a single base that causes a premature in-frame termination codon, can be treated by CMV comprising the sequence of the wild-type gene at the locus of the mutation. As used herein, a CMV has a particular sequence if either strand of the CMV comprises the sequence or comprises a sequence containing ribo-type nucleobase equivalents with uracil bases replacing thymine bases. A frame-shifting deletion of a fragment of an exon or even of a complete exon can be treated by a CMV that differs from the mutated sequence by the presence of a one or two base insertion or deletion such that the correct reading frame is restored downstream of the mutation. Depending on the size of the deletion, gene repair can restore some or all of the normal function of dystrophin in the affected cell. A single-base substitution that affects the splicing of the dystrophin message can be similarly repaired to result in functional dystrophin.

Techniques for the diagnosis of DMD and BMD, as well as the localization and identification of the mutation in the human dystrophin gene responsible for disease, are well known to those skilled in the art. These techniques include the use of antibodies specific to the amino and carboxyl terminals of dystrophin (Bulman et al., American Journal of Human Genetics 48:295-304, 1991; Arahata et al., Journal of Neurological Science 101:148-156, 1991). Such antibody preparations in combination with western blotting can be used to distinguish internal deletions and point mutation that effect reading frame from deletion mutations that do not. The use of RT-PCR with mixtures of multiple exon specific primers that produce PCR fragments of distinguishable diagnostic size allows for the rapid detection of exon deletions in a subject's dystrophin mRNA (Abbs et al., Journal of Medical Genetics 28:304-311, 1991; Beggs et al., Human Genetics 86:45-48, 1990). The sensitivity of RT-PCR diagnosis is sufficient to permit the analysis of dystrophin message from peripheral blood, and identification of the mutation by the sequencing of the product (Roberts, American Journal of Human Genetics 49:298-310, 1991).

The sequence of the homologous region of a CMV of the invention can be selected in accordance with the mutation's location or by the location that is selected for an insertion or deletion to restore the reading frame of the gene. The sequence of the homologous region will have the sequence or its equivalent of a fragment of an exon or an intron that is located within about 25 nucleotides of the exon or of a fragment that bridges an intron and an exon. As used herein the term “flanking intron” refers to the 21 nucleotides of the intron adjacent to an exon. The nucleotide sequence of the exons and flanking intron sequences of the human dystrophin gene are known. Intron sequences not yet published can be obtained by standard techniques well known to those skilled in the art, using the sequence of the exon and the knowledge of the restriction map of the dystrophin gene (the size of the genomic Hind III fragment containing each exon of the dystrophin gene is disclosed in Roberts et al., Genomics 16:536-538, 1993).

CMV may be introduced into solid tissues by intravenous or intraarterial routes for those that are extensively vascularized. Preferred transfection methods, however, involve direct administration to the affected solid tissue that do not deliver the CMV throughout the system in significant amounts. This localizes gene repair to places where it will result in effective treatment while reducing the amount of CMV that is expended and minimizing effects in cells unaffected by the genetic disease or other pathologic condition. Such techniques may include biolistics and electroporation, but direct injection by hypodermic needle is preferred. In particular, administration of a composition localized to affected parenchyma or interstitial spaces proximal to affected tissue are preferred. Alternative techniques include sustained infusion of affected solid tissue by permeable matrices or pumps. Direct administration to localized spaces can be monitored in real time by including an indicator in the composition or determining its distribution at later times.

Methods of treatment according to the invention administer CMV alone or with other agents in a composition in effective amounts. Such treatment of mammalian subjects in need thereof may be (a) therapeutic to treat existing disease and other pathologic conditions and/or (b) prophylactic to prevent or at least reduce the propensity of developing disease and other pathologic conditions. Therapeutically or prophylactically effective amount, as recognized by those of skill in the art, will be determined on a case by case basis. Factors to be considered include, but are not limited to: the tissue-type of the targeted cell and its ability to replicate, synapse, or recombine nucleic acids, the genetic sequence to be altered, the disease or other condition to be treated, and the medical history and status of the subject to be treated. For example, acquired mutations may result in sporadic disease and other pathologic conditions that are easier to treat because gene repair is required in only a few cells.

Chimeric Mutational Vectors (CMV)

Compositions containing at least one chimeric mutational vector (CMV) may be used to deliver the CMV into muscle cells, at least some of which will target the dystrophin gene and direct sequence-specific alterations therein (e.g., insertions, deletions, substitutions of one to six bases). A duplex oligonucleobase consisting of more than 200 deoxyribonucleotides and no nucleotide derivatives is not considered a CMV. Typically, a CMV is characterized by being a duplex oligonucleobase, including ribo-type and deoxyribo-type nucleobases, of lengths between about 20 and about 120 nucleobases or equivalently between about 10 and about 60 Watson-Crick nucleobase pairs.

“Chimeric mutational vectors” are described in U.S. Pat. No. 5,565,350 as a duplex mixed oligonucleobase, which contains at least one strand of ribo-type and deoxyribo-type nucleobases, hybridized to each other. At least one region of contiguous unpaired nucleobases is disposed so that the unpaired region separates the oligonucleobase into a first strand and a second strand. The region of contiguous unpaired nucleobases connects a region of Watson-Crick paired nucleobases of at least 15 base pairs in length, in which the first strand's nucleobases are complementary to the second strand's nucleobases. The first strand may comprise a region of at least three to nine contiguous nucleobases comprised of a 2′-O or 2′-O-Me ribose, which form a hybrid-duplex within the region of Watson-Crick paired bases. Two regions homologous with the sequence of the target gene flank a heterologous region with the alteration. The second strand may contain no nucleobases comprised of a 2′-O or 2′-O-Me ribose. In such a CMV, the first strand may comprise two regions of nucleobases comprised of a 2′-O- or 2 ′-O-Me ribose that form two regions of hybrid-duplex, each hybrid-duplex region having at least four or eight base pairs of length, and an interposed region of at least four or eight base pairs of homo-duplex disposed between the hybrid duplex regions. The interposed region of homo-duplex may consist of between four and 50, or between 30 and 1,000, 2′-deoxyribose base pairs.

“Oligonucleobases” are polymers of nucleobases, which polymer can hybridize by Watson-Crick base pairing to a DNA having the complementary sequence. Nucleobases comprise a base, which is a purine, pyrimidine, or a derivative or analog thereof. Nucleobases include peptide nucleobases, the subunits of peptide nucleic acids, and morpholine nucleobases as well as nucleobases that contain a pentosefuranosyl moiety (e.g., a substituted riboside or 2′-deoxyriboside). A “nucleobase” contains a base, which is either a purine or a pyrimidine or analog or derivative thereof. There are two types of nucleobases. Ribo-type nucleobases are ribonucleosides having a 2′-hydroxyl, substituted 2′-hydroxyl or 2′-halo-substituted ribose. All nucleobases other than ribo-type nucleobases are deoxyribo-type nucleobases. Thus, deoxy-type nucleobases include peptide nucleobases.

“Nucleosides” are nucleobases attached to a pentosefuranosyl sugar, e.g., an optionally substituted riboside or 2 ′-deoxyriboside. Nucleosides can be linked by one of several linkages, which may or may not contain a phosphorus, including substituted phosphodiester bonds (e.g., phosphorothioate or triesterified phosphates). Nucleosides that are linked by unsubstituted phophodiester bonds are termed nucleotides. Other types of heteroatom linkages contain a nitrogen, sulfur, or oxygen.

A oligonucleobase compound has 5′ and 3′ end nucleobases, which are the ultimate nucleobases of the polymer. Nucleobases are either deoxyribo-type or ribo-type. Ribo-type nucleobases are pentosefuranosyl containing nucleobases wherein the 2′ carbon is a methylene substituted with a hydroxyl, substituted oxygen or a halogen. Deoxyribo-type nucleobases are nucleobases other than ribo-type nucleobases and include all nucleobases that do not contain a pentosefuranosyl moiety (e.g., peptide nucleic acids).

An oligonucleobase strand generically includes regions or segments of oligonucleobase compounds that are hybridized to substantially all of the nucleobases of a complementary strand of equal length. An oligonucleobase strand has a 3′ terminal nucleobase and a 5′ terminal nucleobase. The 3′ terminal nucleobase of a strand hybridizes to the 5′ terminal nucleobase of the complementary strand. Two nucleobases of a strand are adjacent nucleobases if they are directly covalently linked or if they hybridize to nucleobases of the complementary strand that are directly covalently linked. An oligonucleobase strand may consist of linked nucleobases, wherein each nucleobase of the strand is covalently linked to the nucleobases adjacent to it. Alternatively a strand may be divided into two chains when two adjacent nucleobases are unlinked. The 5′ (or 3′) terminal nucleobase of a strand can be linked at its 5′-O (or 3′-O) to a linker, which linker is further linked to a 3′ (or 5′) terminus of a second oligonucleobase strand, which is complementary to the first strand, whereby the two strands form one oligonucleobase compound. The linker can be an oligonucleobase, an oligonucleobase or other compound. The 5′-O and the 3′-O of a 5′ end and 3′ end nucleobase of an oligonucleobase compound can be substituted with a blocking group that protects the oligonucleobase strand. Of course, closed circular olignucleotides do not contain 3′ or 5′ end nucleotides. Note that when an oligonucleobase compound contains a divided strand, the 3′ and 5′ end nucleobases are not the terminal nucleobases of a strand.

As used herein the terms 3′ and 5′ have their usual meaning. The terms “3′ most nucleobase,” “5′ most nucleobase,” “first terminal nucleobase,” and “second terminal nucleobase” have special definitions. The 3′ most and second terminal nucleobase are the 3′ terminal nucleobases, as defined above, of complementary strands of a recombinagenic oligonucleobase. Similarly, the 5′ most and first terminal nucleobase are 5′ terminal nucleobases of complementary strands of a recombinagenic oligonucleobase.

More generally, the CMV is a polymer of nucleobases, which polymer hybridizes (i.e., form Watson-Crick base pairs of purines and pyrimidines) in a duplex structure. Each CMV can be divided into a first and a second strand of at least 12 nucleobases and not more than 75 nucleobases. The length of the strands may be each between 20 and 50 nucleobases. The strands contain regions that are complementary to each other. The two strands may be complementary to each other at every nucleobase except the nucleobases wherein the target sequence and the desired sequence differ. At least two non-overlapping regions of at least five nucleobases are preferred.

If the strands are complementary to each other at every nucleobase, the sequence of the first and second strands consists of at least two regions that are homologous to the target gene and one or more regions (the “mutator regions”) that differ from the target gene and introduce the genetic change into the target gene. The mutator region is directly adjacent to homologous regions in both the 3′ and 5′ directions. The two homologous regions may be at least three, six, or 12 nucleobases in length. The total length of all homologous regions may be at least 12, between 16 and 60, or between 20 and 60 nucleobases in length. The total length of the homology and mutator regions together may be between 25 and 45, between 30 and 45, or between 35 and 40 nucleobases. Each homologous region can be between eight and 30, between eight and 15 nucleobases, or about 12 nucleobases long. The mutator region may consist of 20 or fewer, six or fewer, or three or fewer nucleobases. The mutator region can be of a length different than the length of the sequence that separates the regions of the target gene homology with the homologous regions of the CMV so that an insertion or deletion of the target gene results. When the CMV is used to introduce a deletion in the target gene there is no nucleobase identifiable as within the mutator region. Rather, the mutation is effected by the juxtaposition of the two homologous regions that are separated in the target gene. The length of the mutator region of a CMV that introduces a deletion in the target gene is deemed to be the length of the deletion. The mutator region may be a deletion of between one and six nucleobases or between one and three nucleobases. Multiple separated mutations can be introduced by a single CMV, in which case there are multiple mutator regions in the same CMV. Alternatively, multiple CMV can be used simultaneously to introduce multiple genetic changes in a single gene or, alternatively to introduce genetic changes in multiple genes of the same cell. Herein, the mutator region is also termed the heterologous region. When the different desired sequence is an insertion or deletion, the sequence of both strands have the sequence of the different desired sequence.

The 3′ terminal nucleobase of each strand may be protected from 3′ exonuclease digestion. Such protection can be achieved by several techniques now known to these skilled in the art or by any technique to be developed. For example, protection from 3′-exonuclease digestion may be achieved by linking the 3′ most (terminal) nucleobase of one strand with the 5′ most (terminal) nucleobase of the alternative strand by a nuclease-resistant covalent linker, such as polyethylene glycol, poly-1,3-propanediol, or poly-1,4-butanediol. The length of various linkers suitable for connecting two hybridized nucleic acid strands is understood by those skilled in the art. A polyethylene glycol linker having from six to three ethylene units and terminal phosphoryl moieties is suitable (Durand et al., Nucleic Acids Research 18:6353, 1990; Ma et al., Nucleic Acids Research 21:2585-2589, 1993); bis-phosphorylpropyl-trans-4,4 ′-stilbenedicarboxamide may also be used as a linker (Letsinger et al., Journal of the American Chemical Society 116:811-812, 1994; Letsinger et al., Journal of the American Chemical Society 117:7323-7328, 1995). Such linkers can be inserted into the CMV using conventional solid phase synthesis. Alternatively, the strands of the CMV can be separately synthesized and hybridized, and then forming an interstrand linkage with thiophoryl-containing stilbenedicarboxamide as described in patent application WO 97/05284.

The linker can be a single strand oligonucleobase comprised of nuclease-resistant nucleobases (e.g., a 2′-O-methyl, 2′-O-allyl or 2′-F-ribonucleotides). The tetranucleotide sequences TTTT, UUUU and UUCG and the trinucleotide sequences TTT, UUU, or UCG are particularly preferred nucleotide linkers. A linker comprising a tri- or tetra-thymidine oligonucleobase is not comprised of nuclease-resistant nucleobases and such linker does not provide protection from 3′ exonuclease digestion.

Alternatively, modification of the 3′ terminal nucleobase can protect it from digestion by 3′-exonuclease. If the 3′ terminal nucleobase of a strand is a 3′ end, then a steric protecting group can be attached by esterification to the 3′-OH, the 2′-OH or to a 2′ or 3′ phosphate. Suitable protecting group are 1,2-(ω-amino)-alkyldiols or, alternatively, 1,2-hydroxymethyl-(ω-amino)-alkyls. Modifications that can be made include use of an alkene or branched alkane or alkene, and substitution of the ω-amino or replacement of the (ω-amino with an co-hydroxyl. Other suitable protecting groups include a 3′-methylphosphonate, (Tidd et al., British Journal of Cancer 60:343-350, 1989) and 3′-aminohexyl (Gamper et al., Nucleic Acids Research 21:145-150, 1993). Alternatively, the 3′ or 5′ end hydroxyls can be derivatized by conjugation with a substituted phosphorus (e.g., methylphosphonates or phosphorothioates).

Moreover, the 3′-most nucleobase can be made a nuclease-resistant nucleobase to protect the 3′-terminus. Nuclease-resistant nucleobases include PNA nucleobases and 2′ substituted ribonucleotides. Suitable substituents include those disclosed in U.S. Pat. Nos. 5,334,711; 5,658,731; and 5,731,181 and those disclosed in EP 0 629 387 and EP 0 679 657. The 2′ fluoro, chloro, or bromo derivatives of a ribonucleotide or a ribonucleotide having a substituted 2′-O as described in the aforementioned are termed 2′-Substituted Ribonucleotides (e.g., 2′-fluoro, 2′-methoxy, 2′-propyl-oxy, 2′-allyloxy, 2′-hydroxylethyloxy, 2′-methoxy-ethyloxy, 2′-fluoropropyloxy, and 2′-trifluoropropyloxy substituted ribonucleotides; 2′-fluoro, 2′-methoxy, 2′-methoxyethyloxy, and 2′-allyloxy substituted nucleotides). A “nuclease-resistant ribonucleoside” includes 2′-Substituted Ribonucleotides and all 2′-hydroxyl ribo-nucleosides other than ribonucleotides (e.g., ribonucleotides linked by non-phosphate or by substituted phosphodiesters).

CMV may have a single 3′ end and a single 5′ end which are the terminal nucleobases of a strand. Alternatively, a strand may be divided into two chains that are linked covalently through the alternative strand but not directly to each other. Where a strand is divided into two chains, the 3′ and 5′ ends are Watson-Crick base paired to adjacent nucleobases of the alternative strand. In such strands, the 3′ and 5′ ends are not terminal nucleobases. A 3′ end or 5′ end that is not the terminal nucleobase of a strand can be optionally substituted with a steric protector from nuclease activity as described above. Alternatively, a terminal nucleobase of a strand is attached to an nucleobase that is not paired to a corresponding nucleobase of the opposite strand and is not a part of an interstrand linker. It has a single “hairpin” conformation with a 3 or 5′ overhang. The unpaired nucleobase and other components of the overhang are not regarded as a part of a strand. The overhang may include self-hybridized nucleobases or non-nucleobase moieties (e.g., affinity ligands or labels). In a CMV having a 3′ overhang, the strand containing the 5′ nucleobase may be composed of deoxy-type nucleobases only, which are paired with ribo-type nucleobase of the opposite strand. For a CMV having a 3′ overhang, the sequence of the strand containing the 5′ end nucleobase is the different, desired sequence and the sequence of the strand having the overhang is the sequence of the target gene.

The linkage between the nucleobases of the strands of a CMV can be any linkage that is compatible with hybridization of the CMV to its target sequence. Such sequences include the conventional phosphodiester linkages found in natural nucleic acids. The organic solid phase synthesis of oligonucleobases is described in U.S. Patent No. Re 34,069.

The internucleobase linkages can also be substituted phosphodiesters (e.g., phosphoro-thioates, substituted phosphotriesters). Alternatively, non-phosphate, phosphorus-containing linkages can be used. U.S. Pat. No. 5,476,925 describes phosphoramidate linkages. The 3′-phosphoramidate linkage (3′-NP(O-)(O)0-5′) is well suited for use in CMV because it stabilizes hybridization compared to a 5′-phosphoramidate. Non-phosphate linkages between nucleobases can also be used. U.S. Pat. No. 5,489,677 describes internucleobase linkages having adjacent nitrogen and oxygen heteroatoms, and their synthesis. Another preferred linkage is 3′-ON(CH₃)CH₂-5′ (methylenemethylimino). Other linkages suitable for use in CMV are described in U.S. Pat. No. 5,731,181. Nucleobases that lack a pentosefiranosyl moiety and are linked by peptide bonds can also be used. Such so-called peptide nucleic acids (PNA) are described in U.S. Pat. No. 5,539,082; methods for making PNA/nucleotide chimera are described in patent application WO 95/14706. The 2′ position end of the intemucleobase linkage can be modified (Freier & Altmann, Nucleic Acids Research 25:4429-4443, 1997).

Formulation of the Compositions

A polymer (e.g., polyethylene glycol or PEG, polyethylenimine or PEI) can be included in the composition. They could have an average molecular weight of greater than about 500 daltons, preferably greater than between about 10 kd and more preferably about 25 kd (mass average molecular weight determined by light scattering). The upper limit of suitability is determined by the toxicity and solubility of the polymer, but molecular weights greater than about 1.3 Md are possibly less suitable. Alternatively, inert polymeric materials could be formed into nanospheres or microspheres as transfection agents (cf. Leong et al., Journal of Controlled Release 53:183-193, 1998; Baranov et al., Gene Therapy 6:1406-1414, 1999).

The use of high molecular weight PEI as a carrier to transfect a cell with DNA is described in Boussif et al., Proceedings of the National Academy of Sciences 92:7297-7301, 1995. A CMV carrier complex can be formed by mixing an aqueous solution of CMV and a neutral aqueous solution of PEI at a ratio of between about 4 and about 9 PEI nitrogens per CMV phosphate, preferably the ratio is about 6. The complex can be formed, for example, by mixing a 10 mM solution of PEI, at pH 7.0 in 0.15 M NaCl with CMV at a final concentration of between 100 and 500 nM CMV.

A ligand can also be included in the composition. Suitable ligands are those that specifically bind receptors in clathrin-coated pits, transferrin, nicotinic acid, a-bungarotoxin, camitine, insulin, and insulin like growth factor-1 (IGF-1). In an alternative embodiment, the ligand contain glucosyl moieties, such as glucose. For example, a 1:1 mixture of glucosylated PEI having a ratio of between about 0.4 and about 0.8 glucose moieties per nitrogen and unmodified PEI can be used. The mixture is used in a ratio of between 4 and 9 PEI nitrogens per CMV phosphate, preferably the ratio of CMV phosphate to nitrogen is about 1:6. PEIs having a mass average molecular weight of 25 kd and 800 kd are commercially available from Aldrich Chemical Co., Catalog No. 40,872-7 and 18,197-8, respectively. The optimal ratio of ligand to polyethylene subunit can be determined by fluorescently labeling the CMV and injecting flourescent CMV/molecular carrier/ligand complexes directly into the tissue of interest and determining the extent of fluorescent uptake according to the method of Kren et al., Hepatology 25:1462-1468, 1997. Furthermore, a basic protein (e.g., histone H1) can be substituted as a polycationic carrier.

Transfection agents that at least in part condense the CMV may be used. Alternatively, transfection agents like lipids may form liposomes or other structures that encapsulate the CMV. Many neutral and charged lipids, sterols, and other phospholipids to make lipid carrier vehicles are known.

Synthetic lipids or purified lipid biological preparations, e.g., soybean oil (Sigma) or egg phosphatidyl choline (EPC) (Avanti Polar Lipids) can be used. Other lipids that are useful in the preparation of lipid nanospheres and/or lipid vesicles include neutral lipids, e.g., dioleoyl phosphatidylcholine (DOPC) and dioleoyl phosphatidyl ethanolamine (DOPE); anionic lipids, e.g., dioleoyl phosphatidyl serine (DOPS); and cationic lipids, e.g., dioleoyl trimethyl ammonium propane (DOTAP), dioctadecyldiamidoglycyl spermine (DOGS), dioleoyl trimethyl ammonium (DOTMA), and DOSPER (1,3-di-oleoyloxy-2-(6-carboxy-spermyl)-propyl-amide tetraacetate, commercially available from Boehringer-Mannheim. Additional examples of lipids that can be used in the invention can be found in Gao & Huang (Gene Therapy 2:710-722,1995). Saccharide ligands can be added in the form of saccharide cerebrosides, e.g., lactosylcerebroside or galactocerebroside (Avanti Polar Lipids). DPPC (dipalmitoyl phosphatidylcholine) can be incorporated to improve the efficacy and/or stability of delivery. FUGENE 6, LIPOFECTAMINE, LIPOFECTIN, DMRIE-C, TRANSFECTAM, CELLFECTIN, PFX-1, PFX-2, PFX-3, PFX-4, PFX-5, PFX-6, PFX-7, PFX-8, TRANSFAST, TFX-10, TFX-20, TFX-50, and LIPOTAXI lipids are proprietary sources of lipid.

Lipid nanospheres can be constructed by the following process. A solution of phospholipids in organic solvent is added to a small test tube and the solvent removed by a nitrogen stream to leave a lipid film. A lipophilic salt of CMV is formed by mixing an aqueous saline solution of CMV with an ethanolic solution of a cationic lipid. The cationic species can be in about 4 fold molar excess relative to the CMV anions (phosphates). The lipophilic CMV salt solution is added to the lipid film, vortexed gently followed by the addition of an amount of neutral lipid equal in weight to the phospholipids. The concentration of CMV can be up to about 3% (w/w) of the total amount of lipid. After addition of the neutral lipid, the emulsion is sonicated at 4° C. for about 1 hour until the formation of a milky suspension with no obvious signs of separation. The suspension is extruded through polycarbonate filters until a final diameter of about 50 nm is achieved. The CMV-carrying lipid nanospheres can then be washed and placed into a pharmaceutically acceptable carrier or tissue culture medium. The capacity of lipid nanospheres is about 2.5 mg CMV/500 μl of a nanosphere suspension.

A lipid film is formed by placing a chloroform methanol solution of lipid in a tube and removing the solvent by a nitrogen stream. An aqueous saline solution of CMV is added such that the amount of CMV is between 20% and 50% (w/w) of the amount of lipid, and the amount of aqueous solvent is about 80% (w/w) of the amount of lipid in the final mixture. After gentle vortexing the liposome-containing liquid is forced through successively finer polycarbonate filter membranes until a final diameter of about 50 nm is achieved. The passage through the successively finer polyearbonate filter results in the conversion of polylaminar liposomes into unilaminar liposomes (i.e., lipid vesicles). The lipid nanospheres can then be washed and placed into a pharmaceutically acceptable carrier. About 50% of the added CMV can be entrapped in the vesicle's aqueous core.

A variation of the basic procedure comprises the formation of an aqueous solution containing a PEI/CMV condensate at a ratio of about 4 PEI imines per CMV phosphate. The condensate can be particularly useful when the liposomes are positively charged, i.e., the lipid vesicle contains a concentration of cations of cationic lipids such as DOTAP, DOTMA or DOSPER, greater than the concentration of anions of anionic lipids such as DOPS. The capacity of lipid vesicles is about 150 μg CMV per 500 μl of a lipid vesicle suspension.

Lipid vesicles may contain a mixture of the anionic phospholipid, DOPS, and a neutral lipid such as DOPE or DOPC; negatively charged phospholipids that can be used to make lipid vesicles include dioleoyl phosphatidic acid (DOPA) and dioleoyl phosphatidyl glycerol (DOPG). For example, the neutral lipid may be DOPC and a ratio of DOPS:DOPC between about 2:1 and about 1:2, preferably about 1:1. The ratio of negatively charged to neutral lipid is preferably greater than about 1:9 because the presence of less than 10% charged lipid results in instability of the lipid vesicles because of vesicle fusion.

An optional additive to the composition is an insoluble indicator that will not diffuse a substantial distance in solid tissue from the site of injection. For example, a signal-generating particle mixed into the composition with indicate the injection track. Gene repair and/or a change in physiological resulting from gene repair can then be correlated with localization of the CMV introduced into cells. The signal can be a fluorophore, radioisotope, other emitters of electromagnetic radiation, colloidal metal, contrast agent for ultrasound or electromagnetic radiation, chromagen, or be generated by an enzyme attached to the particle (e.g., alkaline phosphatase, horseradish peroxidase). Similarly, entry into cells can be determined by labeling the CMV, and then visualizing the label or comparing the amount of label in separated extracellular and intracellular fractions. Placement of CMV in situ may be guided by soluble or insoluble signals (e.g., fluorophores, radiochemicals, other emitters of electromagnetic radiation, contrast agents) and ultrasonography/radiography, or visualized with fiber optics.

At least some of the CMV and optional agents of the composition may self-assemble upon mixing. They may associate by interactions that are covalent (e.g., linkages with an amino or thiol reactive group, photo adducts) or non-covalent (e.g., hydrogen bonding, electrostatic or hydrophobic forces). The degree of association may be assessed by techniques such as, for example, fluorescence quenching or transfer, light polarization or scattering, electrophoretic mobility, size-exclusion chromatography, and electron microscopy.

Canine Model of Muscular Dystrophy

A composition comprising a CMV packaged in FuGENE™ 6 lipid was introduced into an affected cell and produced dystophin protein containing exon 7 epitopes. The invention further encompasses the use of alternative lipid carriers that are equivalent to FuGene™ 6 lipid, now known or to be developed. Naked CMV (i.e., introduced into an affected cell without transfection agents like lipids, viral particles, DEAE-dextran, salt and polymeric precipitants, etc.) are not effective in this embodiment of the invention. But it is well within the skill of the art to determine under which circumstances naked CMV could be effectively used for gene repair (e.g., the mdx mutation exemplified below).

Correction of the GRMD point mutation, as detected at the mRNA, protein, and genomic DNA levels, was found up to 11 months after a single treatment with the CMV. To facilitate the analysis of the GRMD model, an exon 7-specific antibody against the portion of the protein deleted by the GRMD mutation provided a unique reagent for discriminating patterns of dystrophin protein expression resulting from successful gene repair to that produced by alternative processing of the mRNA. The critical importance of exon-specific antibodies for unequivocal identification of wild-type dystrophin in muscle fibers has been demonstrated in human myoblast therapy trials. At 11 months post-injection, detectable quantities of normal sized dystrophin were localized in multiple regions within the treated cranial tibialis muscle using the MANEX7B antibody. These results were obtained by both western blot and immunohisto-chemical analyses using the MANEX7B antibody. We estimate that the level of gene repair approaches, but does not exceed, 1% in our studies based on comparative levels of RT/PCR product from the exon 7-deleted mRAN produced by the GRMD allele in the nine-week biopsy sample. To clarify these analyses, RT/PCR primers were specifically selected to discriminate the mutant mRAN and corrected mRNA species from alternately spliced products. Precise quantitative estimates of the level of reversion have proven difficult due to the inherent AT-rich nature of the intron portion of this splice junction, which renders a quantitative method such as allele-specific primer discrimination problematic at best. Thus, we have been limited to qualitative differences rather than quantitative differences between the mRNA/genomic results from the tissues treated with the two CMV used in these experiments versus untreated tissue from the same animal. It is of interest to note that RT/PCR of RNA extracted from the necropsy samples from the right limb treated with the chimera without FuGENE™ 6 lipid failed to produce any detectable exon 7-containing dystrophin mRNA. This is in contrast to the localization seen in both frozen sections taken from the small biopsy sample taken at six weeks for the in situ RT/PCR as well as the immunohistochemistry of the six-week sample. Based on this difference, we believe that the initial frequency of gene repair for the two limbs favored delivery using a carrier vehicle of FUGENE™ 6 lipid for sustained inclusion in nascent dystrophin mRNA of epitope expression of exon 7.

Murine Model of Muscular Dystrophy

The mdx mouse strain has a point mutation in the dystrophin gene, the consequence of which is a muscular dystrophy due to deficiency of dystrophin in skeletal muscle. As a test of the feasibility of CMV-mediated gene therapy for muscular dystrophies, a CMV termed MDX1 was designed to induce correction of the point mutation in the dystrophin gene in mdx mice. Two weeks after direct injection of MDX1 into muscles of mdx mice, dystrophin expression was detected in clusters of muscle fibers by immunohistochemical analysis. None of these dystrophin-positive fibers were so called “revertant” fibers (which appear spontaneously in mdx muscle) as characterized by antibodies directed against the protein products of specific exons of the dystrophin gene. Furthermore, injection of control CMV did not yield any dystrophin-positive fibers. Immunoblot analysis of dystrophin immunoprecipitated from MDX1-injected muscles revealed a single band corresponding to full-length dystrophin. No dystrophin was detected when muscles injected with control CMV were subjected to the same analysis. These results provide the foundation for further studies of CMV-mediated gene therapy as a novel therapeutic approach to muscular dystrophies and other genetic disorders of muscle.

The invention is used to correct a point mutation in the dystrophin gene in the mdx mouse. The mdx mouse has a point mutation at nucleotide position 3185 in the dystrophin gene that produces a stop codon in exon 23 (Yoon et al., Proceedings of the National Academy of Sciences USA 93:2071-2076, 1996). As a result, there is no dystrophin produced in skeletal muscle of these mice and the muscle fibers undergo necrotic degeneration as in DMD. Direct injection into skeletal muscle of a CMV designed to correct the point mutation resulted in the expression of functional dystrophin in muscle fibers around the site of injection. Lipid was not required in this embodiment of the invention.

### EXAMPLES

The following examples are provided for illustrative purposes only and are not to be construed as limiting the invention's scope in any manner.

Canine Model of Muscular Dystrophy

### Correction of the GRMD Mutation Requires Lipid

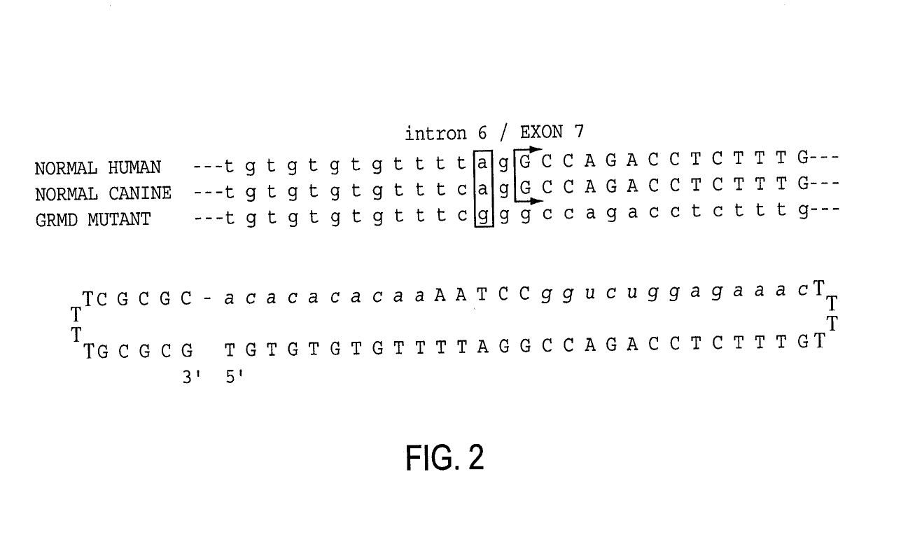

A diagram of the basis of the sequence of the CMV is shown in FIG. 2. The CMV is composed of a five-base segment of DNA which defines the complement of the wild-type coding strand sequence at the splice acceptor site of intron 6 of the canine dystrophin gene (Sharp et al., Genomics 13:115-121, 1992) flanked by complementary segments of 0-methyl-RNA (10-13 residues), two hairpin turns composed of four dT bases, a 3′ GC clamp segment, and a 5′ complementary DNA strand which extends across either end of the two O-methyl-RNA segments. The native structure of such a molecule is believed to be a hairpin (Ye et al., Molecular Medicine Today 4:431-437, 1998). Comparison of the nucleotide sequence of the GRMD mutation and the CMV sequence predicts that the mismatch should be corrected by CMV-mediated gene repair in a treated dog.

An affected male (six weeks of age) from a litter born at the University of Missouri colony was selected for this study. All animals are maintained in the University of Missouri Vivarium according to ACUC and NIH guidelines for the use of animals in research. At 13 months of age, disease symptoms warranted euthanizing the animal. All surgical biopsy and necropsy samples from treated sartorial compartment muscles as well as the left triceps were collected, wrapped in aluminum foil, and snap-frozen in liquid nitrogen. To determine if gene repair mediated by CMV could be used to correct the mutation that causes GRMD, a six week-old affected male was selected for study.

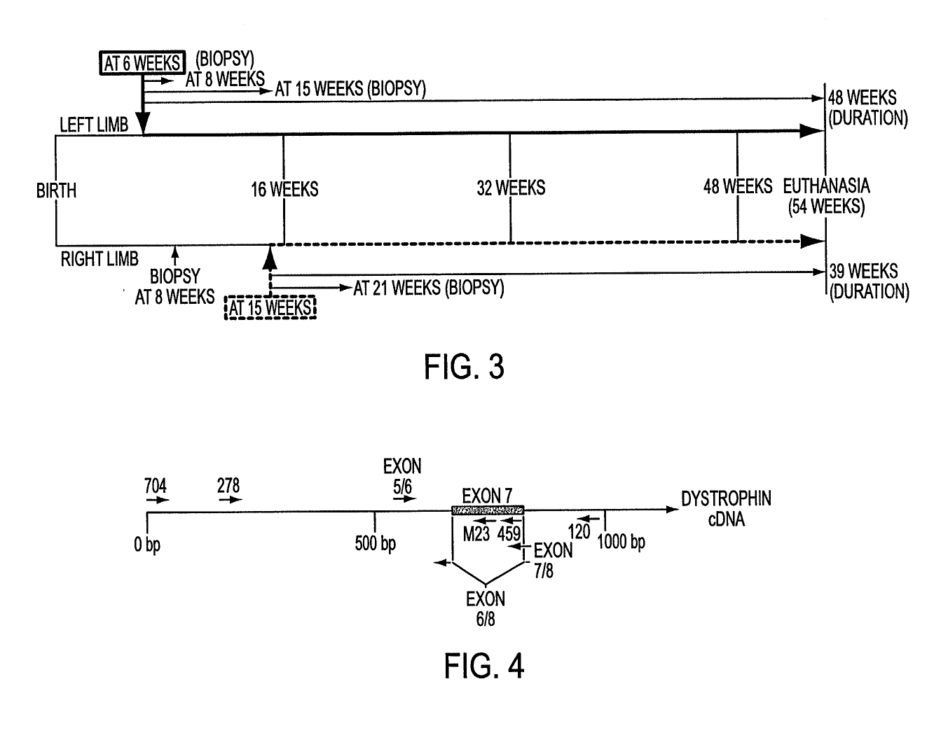

A timeline diagram of the experimental procedures performed on the GRMD affected male is found in FIG. 3. At six weeks of age (time point A), CMV designed to correct the GRMD mutation (200 μg from BioSource) was mixed with 200 μg of calf thymus histone Hi (Sigma) and packaged in FuGENE™ 6 lipid plus OPTIMEM media (LTI) in a final volume of 5.0 ml. Proprietary FUGENE™ 6 lipid is commercially available from Roche Diagnostics (http://biochem.roche.com/techserv/fugene.htm); it is a proprietary blend of lipids and other components supplied in 80% ethanol, sterile filtered, and packaged in polypropylene tubes. The injectate also contains 7.5 μl/ml of fluorescent microspheres (Molecular Probes) to mark the site of injections.

After surgical exposure of the sartorial compartment, the full 5.0 ml was injected into the cranial tibialis compartment of the left limb using 50 separate injections of 100 μl each. Surgical biopsy samples were taken and snap-frozen in liquid nitrogen at 2 (time point B) and 9 (time point C) weeks post-injection (Bartlett et al., Nature Biology Short Reports 9:163-164, 1998) and at necropsy at 11 months (point E) post-injection from the left leg. A biopsy of control untreated triceps muscle was removed for RNA, western, and immunohistochemical analyses prior to injection.

To determine whether FuGENE™ 6 lipid is required to correct the GRMD mutation, additional CMV from Kimeragen was injected into the contralateral limb during the surgical procedure for the 9 week biopsy (time point C). A biopsy was also taken from the contralateral limb at 6 weeks post-injection (time point D). The protocol for treatment was the same as that used for the left leg with the lone exception that FuGENE™ 6 lipid was not included in the injectate. Force generation studies (diagonal arrows) were performed at the three indicated times. The entire cranial tibialis, long digital extensor and triceps muscles (left and right) were removed at necropsy at 13 months of age when the animal was euthanized (time point E) due to progressive disease complications.

To summarize the results obtained and further discussed below, we found that a lipid carrier was required for sustained inclusion in nascent dystrophin mRAN of the epitope encoded by exon 7. A composition that did not include FuGENE™ 6 lipid was ineffective and produced no dystrophin protein containing exon 7 epitopes.

### RT/PCR Analysis Detects Normal-Sized Dystrophin mRNA in Treated Skeletal Muscle

To investigate whether therapy with CMV that corrected the GRMD mutation would produce a change in the pattern of expression of functional dystrophin in GRMD muscle, total RNA was isolated from frozen sections of biopsy and necropsy material taken at various timepoints after treatment. To isolate RNA, about 10-20 serial frozen 20 μm thick sections from the same segments of muscle used for parallel analyses by western blotting and immuno-histochemistry (see below) were made and stored at −80° C. in separate RNAse-free tubes.

Total RNA was isolated using an RNAEASY kit (Promega). RT/PCR was performed using 5′ primer (544) and 3′ primers (704 and 120) that bracket exon 7 of the canine dystrophin mRNA (Sharp et al., Genomics 13:115-121, 1992). The primers used in this analysis are shown in FIG. 4 positioned relative to the respective sequence. The direction of the arrows indicate 5′ primers (right pointing arrows) and 3′ primers (left pointing arrows). RT/PCR products were separated on ethidium-stained 1% agarose gels with normal product at 1058 bp and mutant product at 929 bp.

While suggestive levels of normal-sized dystrophin RT/PCR product containing exon 7 were seen in the 2 week sample, the results from the 9 week sample demonstrated that at least as much product from normal-sized mRNA was present in the biopsy as the mutant mRNA. Confirmation of the presence of exon 7 in the PCR product was by sequencing and re-PCR with an exon 7-specific 3′ primer and the original 5′ primer. Moreover, analysis of a necropsy sample from the left limb (FuGENE™ 6 lipid-treated sample) taken at 11 months reveals that the predominant RT/PCR product was of normal size. Since the level of mutant mRNA is <1% of normal in muscle of GRMD dogs and not visible on a northern blot, we conclude that the frequency of gene repair in these studies produced a similar modest level of normalized dystrophin mRNA.

### Gene Repair of GRMD Mutation Corrects Endogenous Sequence in Affected Tissue

To confirm that the invention had actually corrected the mutation, genomic DNA was isolated from additional serial frozen sections and its nucleotide sequence was determined. Genomic DNA was isolated from twenty 20 μm frozen sections from untreated tricep muscle, treated cranial tibialis (CT), and normal CT muscles using a commercial kit from Qiagen. PCR of genomic DNA was performed using intronic primers that bracket exon 7 in the canine dystrophin gene (Bartlett et al., American Journal of Veterinary Research 57:650-654, 1996).

The GRMD mutation produces a novel Sau96 recognition site such that digestion of the 310 bp genomic PCR product is diagnostic of the mutant allele. Thus, to enrich for corrected GRMD sequences that could be detected by PCR amplification, all samples were digested with Sau96 to deplete GRMD alleles that had not undergone gene repair: reactions were stopped after 10 cycles of PCR with bracketing primers, submitted to digestion with Sau96, extracted with phenol/chloroform and precipitated from ethanol. The Sau96-digested samples were amplified for another 25 cycles and 310 bp bands from each were separately ligated into the TA cloning vector pCR1 (Invitrogen). After analytical digestion with Sau96, all clones from the untreated triceps muscle cut to completion which is indicative of the GRMD allele and all clones from normal CT muscle were resistant to digestion. Clones were sequenced using an Applied BioSystems automated sequencer at the University of Miami Cancer Center DNA Core. Examination of 50 clones from the left CT muscle identified three that demonstrated a pattern of digestion with Sau96 indistinguishable from that obtained from a canine muscle sample for the normal allele. These three clones were sequenced and each contained the corrected sequence containing the functional splice acceptor site.

In no case were we able to detect a two-base change in these clones of PCR products. This may be due to a bias imparted by the analysis of only Sau96 resistant clones or to a lower efficiency of changing two bases as opposed to one. It was also possible that cloning with this technique may have selected for only single-base changes due to the inclusion of only the 3′ base change within the Sau96 recognition site. Screening a larger number of clones (e.g., 600 to 1000) by sequencing might have detected a 5′ base change.

### Quantitation of Gene Repair Events by RT/PCR

Accurate quantitative estimates of CMV-mediated changes in endogenous sequences have proven difficult due to the inherent AT-rich nature of the intron portion of this splice junction, which renders a quantitative method such as allele-specific primer discrimination problematic at best. We have used quantitative RT/PCR to demonstrate that inclusion of exon 7 in the dystrophin mRNA from treated GRMD muscle exceeded 10% of normal levels in an isolated sample.

Total RNA were extracted from frozen sections collected separately from tissue harvested at biopsy or necropsy and stored frozen at −80° C. Control and experimental muscle tissue sections were extracted using the TRIREAGENT RNA isolation kit (Molecular Research Center). RNA concentrations were determined by spectrophotometry and their integrity was verified by electrophoretic analysis. The RT/PCR reaction was performed according to the manufacturer specifications using the C. therm RT/PCR kit (Roche) and sequence-specific RT/PCR primers which bracketed the GRMD mutation, a deletion of exon 7 from the mRNA due to a point mutation in the consensus splice acceptor site of intron 6 (Sharp et al., Genomics 13:115-121, 1992). Primer 278 (canine dystrophin forward) was from exon 1 beginning with the start codon, 5′-ATGCTTTGGTGGGAAGAAGTAGAG-3′ (SEQ ID NO:9) and primer 120 (canine dystrophin reverse) was from exon 8 at positions 990-967 in the cDNA, 5′-GTCACTTTAGGTGGCCTTGGCAAC-3′ (SEQ ID NO: 10).

Nested canine-specific primers located at 538-568 bp spanning the exon 5/6 junction and at 874-846 spanning the exon 7/8 junction were used to specifically amplify the normal canine cDNA in the dilution series. The forward primer spanning the exon 5/6 junction was 5′-GATTTGGAATATAATCCTCCA(TGGCAGGTC-3′ (SEQ ID NO:13) and the reverse primer spanning the exon 7/8 junction was 5′-AGTGGTGGCAACATCTTCAGGATCAA-3′ (SEQ ID NO: 14). The sequence of all canine dystrophin primers was determined by sequencing two clones obtained from RT/PCR of normal canine skeletal muscle RNA using dystrophin primers based on the human cDNA beginning at the first base (5′ primer) and ending at position 1505 bp (3′ primer).

To insure constant input mRNA from each sample, all total RNA samples were first normalized using this primer set for the housekeeping gene GAPDH with the forward primer 5′-ATGATGACATCAAGAAGGTGGTGAAGC-3′ (SEQ ID NO:11) and the reverse primer 5′-TCTCTCCTCCTCGCGTGCTCTTGCTG-3′ (SEQ ID NO: 12). GAPDH gene transcripts were amplified using parallel RT/PCR reactions with a constant sample volume (2 μl) and quantitated using a standard curve generated from normal muscle total RNA. Thereafter, all total RNA input values for dystrophin RT/PCR reactions were normalized to the values generated for GAPDH quantitation. All values for GAPDH production fell within a 2-fold range thus minimizing the range of input template volumes for all dystrophin quantitations. The GAPDH and dystrophin products were not co-amplified in the RT/PCR reactions because several artifactual bands were produced by the presence of both sets of primers which prevented quantitation of either in the LIGHTCYCLER thermal cycler (Roche).

RT/PCR was performed using the following program in a Perkin Elmer 480 PCR machine: cDNA synthesis (30 min at 53° C. and denaturation at 95° C. for 5 min), then 20 cycles of PCR amplification using: denaturation at 95° C. for 30 sec, annealing at 56° C. for 45 sec, and extension at 68° C. for 60 sec. A final polishing step of 72° C. for 7 min was performed. A nested 5′ primer located at 538-568 bp spanning the exon 5/6 junction and primer 120 were combined to re-amplify the various dystrophin RT/PCR products to confirm predicted sizes of a 452 bp product from normal dys mRNA and 333 bp product from GRMD mRNA reflecting the deletion of exon 7. The products were submitted to a melting curve analysis using the LIGHTCYCLER thermal cycler, and a 3° C. difference was noted (i.e., GRMD Tm=81° C., Normal Tm=84° C.).

Alternatively, real-time PCR amplification was performed with a fluorescent LIGHT CYCLER thermal cycler equipped with software that follows the PCR reaction “on-line” step-by-step through all the phases. It also provides us with melting curve analysis and calculations of melting temperature [Tm] of the PCR product. Moreover, quantitation of experimental samples is provided when a standard concentration curve is included in the assay. RT/PCR product from normal muscle was used to generate a standard concentration curve beginning with a 1:10 dilution (0.1×) and through successive 1:10 dilutions down to 1:10⁵ (0.00001×).

Quantitative PCR was performed in a LIGHTCYCLER thermal cycler in a final volume of 20 μl containing 2 μl of ready-to-use reaction mix 10 (X). DNA Master SYBR Green I (Roche) was preincubated 5 min at room temperature with 0.55 μg of TAQSTART antibody (Clontech), 3 mM MgCl₂, 0.5 μM of each primer, and 2 μl of either the RT/PCR dilution series or a 1:200 dilution of the experimental RT/PCR sample as template. The program to amplify exon-specific products used an initial denaturation step of 95° C. for 20 sec to inactivate the Taq antibody; 65 cycles of denaturation at 96° C. for 5 sec/annealing at 63° C. for 4 sec/extension at 72° C. for 30 sec; and acquisition of fluorescence for all samples after heating to 82° C. Thus, the fluorescence is acquired above the Tm of the mutant product (81° C.) to insure that the normal product in all samples is measured by fluorescence quantitation. The expected size for the normal dystrophin amplification product is 334 bp.

After each quantitative PCR was completed, a melting curve analysis was performed by heating to 96° C., and cooling to 35° C. at 20° C./sec followed by heating to 96° C. at much slower rate (0.1° C./s) and acquiring fluorescence continuously. Identity of PCR products was verified by melting temperature [Tm] and electrophoresis on 1% agarose gel.

All measurements were taken at 83° C. to measure the exon 7/8-specific product. The cycle number in which fluorescent signal begins to accumulate is inversely proportional to the starting concentration of exon 7-containing dystrophin cDNA in the template sample. PCR products produced in the indicated samples all contain the expected 330 bp product when run on a 2% NUSIEVE agarose (FMC) gel and stained with ethidium bromide. Since all reactions are taken to equilibrium (completion) during the course of the real-time PCR, the standard curves do not relect a gradient of concentration when run on this gel. Of critical importance to note, the sample from the affected, untreated tissue, RTCT 2 weeks, contained no product.

Moreover, the excellent agreement of the concentration estimates for the standard curves with the expected values, and the production of the appropriately-sized products, demonstrates that the 3′ primer used to detect the exon 7/8 junction in the cDNA from the original RT/PCR products provided the appropriate specificity for detecting the presence of inclusion of exon 7 in the cDNA from the experimental samples.

### In situ RT/PCR of Treated Skeletal Muscle Localizes Gene Repair Events

To determine what the pattern of distribution of gene repair was in the injected muscle, we performed in situ RT/PCR on frozen sections from normal, GRMD muscle, and the 6 week injected sample from the right leg. Frozen sections of muscle from normal, GRMD mutant, and GRMD injected muscle were prepared on SUPERFROST slides (Fisher Scientific) using a Leica 3000 cryomicrotome. After overnight fixation in 10% buffered formalin, slides were rinsed twice in fresh PBS, then digested for 17 min in 5 μg/ml pepsin. This permitted infusion of RT/PCR reagents into the extensively fixed tissue. Slides were then rinsed in two changes of fresh PBS, treated with RNAse free-DNAase to remove nuclear DNA as template from the subsequent RT/PCR reaction, and finally rinsed in four changes of fresh PBS. The slides were covered using in situ chambers (RPI, Sci.). Using RT/PCR 3′ primers from within exon 7 (459 and M23) and a 5′ primer which spans intron 6 (354) in genomic DNA (begins in exon 5 and ends in exon 6), RT/PCR was performed using the Roche/Boehringer Mannheim single tube TITAN RT/PCR kit (i.e., a master mix containing the single enzyme TthI for performing both RT and PCR in a single tube) in the presence of dATP-biotin to label all PCR products with biotin.

Streptavidin conjugated with alkaline phosphatase (AP) and ELF-97 fluorochrome (Molecular Probes) were used to localize the biotinylated PCR products. Thus, after RT/PCR, slides were rinsed twice with fresh PBS and then treated at room temperature with streptavidin alkaline phosphatase derivative to bind the in situ biotinylated PCR product. Then the slides were again rinsed with three changes of fresh PBS, followed by 5 min exposure to the ELF-97 fluorochrome according to the supplier's instructions. ELF-97 fluorochrome is a soluble, pale blue fluorescing phosphate in its original form but upon cleavage by AP, a precipitate is produced that is brightly yellow-green in fluorescence at the sites of biotin incorporated into PCR product. A DAPI long-pass filter (Leitz) was used to visualize this signal from biotin.

Examination of negative control sections from GRMD triceps muscle obtained via biopsy prior to injection revealed complete absence of exon 7 across the entire section. But positive control sections from normal canine muscle expressed exon 7 across the entire section. Experimental sections from injected GRMD muscle had modest localization of exon 7 across the entire section particularly near to fluorescent microspheres indicating proximity to sites of injection. At high magnification, the injected samples show discrete localization of exon 7-containing dystrophin mRNA at the periphery of fibers where one would expect the myonuclei to be located. These results suggest that modest reversion occurred in multiple nuclei proximal to the sites of injection.

### Preparation of Exon 7-Specific Monoclonal Antibodies

Frozen sections of 6 μm of thickness from untreated tricep muscle, injected cranial tibialis (CT) muscle, and normal CT muscle were made using a Leica 3000 cryomicrotome and applied to SUPERFROST slides. Primary monoclonal antibodies against dystrophin included a commercially available antibody specific for the carboxy-terminal region (Novacastra) or exon 7-specific as described below. Primary antibody was applied directly to slides at 1:20 dilution in the presence of 5% normal goat serum, while a goat anti-mouse secondary antibody labeled with FITC (Sigma or Jackson Immunesciences) was used to provide a fluorochrome for localization of dystrophin. Slides were counter-stained for 10 min with DAPI (Sigma) at 15 μg/ml. Images were captured using ⅛ sec pixel accumulation as TIFF files with an Optronics cooled CCD camera and ScionImage frame-grabber installed in a PowerMac G3 and converted to Photoshop JPEG files for printing on an HP 5M Color Laserprinter.

Initial western blotting and histochemical analysis of the 2 and 9 week samples obtained from tissue of the left limb, as well as the 6 week sample taken from the right limb using a commercially available carboxy-terminal dystrophin antibody (Novacastra), suggested no detectable increase in dystrophin protein and modest evidence of dystrophin positive fibers located in the region of the injection site marked by fluorescent microspheres. But the levels were no higher than background when compared to uninjected sample from the triceps muscle taken from the same animal prior to therapy. To increase specificity in the immunological analyses, an exon 7-specific antibody was generated for use.

Dystrophin cDNA (cf27 in pUC plasmid from Prof. Kay Davies) was digested with BamHI and NcoI. The 1640 bp fragment from exon 4 to exon 16 was purified and ligated into pMW172 cut with the same restriction enzymes. After electroporation into E. coli BL21(DE3), protein expression was induced by 0.4 mM IPTG for 3 hr. Inclusion bodies were isolated by sonication and extracted sequentially with increasing concentrations of urea (2M, 4M, 6M and 8M in PBS). A 5 μg/ml solution of recombinant protein in 8M urea was used to immunize BALB/c mice and monoclonal antibodies were produced by the hybridoma fusion method. Supernatants were screened by ELISA with recombinant proteins and positive wells (110 out of 288) were further tested for reaction with both native dystrophin (immunolocalization at muscle membrane) and denatured dystrophin (binding to an about 427 kd band on western blots of human muscle proteins). Fourteen wells that passed this screening process were cloned twice by limiting dilution to establish the hybridoma lines. Ig subclass was determined using a mouse isotyping kit (Serotec). Control blots with normal human lung showed that only one mouse mAb (MANEX1011E) cross-reacted with utrophin.

Fourteen mAbs raised against a fragment of dystrophin encoded by exons 4-16 were mapped by western blotting with fragments produced by PCR. Exon 7-specific mabs, for example, recognize an exon 7-16 fragment, but do not recognize exon 8-16 or any smaller fragment. This shows that exon 7 is essential for binding, and we may be confident that the exon 7-specific mAbs will not recognize “revertant” dystrophins lacking exon 7.

Subconstructs of the pMW172:exon 4-16 construct were produced by PCR for epitope mapping. Forward primers with added BamHI sites were synthesized by the Human Genome Mapping Resource Center (Cambridge, UK) as follows: exon 6 (ctcggatcccaggtcaaaaatgtaatg, SEQ ID NO:15), exon 7 (ggggatccaggccagacctatttgac, SEQ ID NO:16), exon 8 (ggggatccgatgtt-gataccacctatc, SEQ ID NO:17), exon 10 (ggggatcccatttggaagctcctga, SEQ ID NO:18) and exon 12 (ggggatcccatagagttttaatggatctc, SEQ ID NO: 19). The reverse primer in the pMW172 sequence was gttattgctcagcggtggcagcag (SEQ ID NO:20). PCR products were digested with BamHI and EcoRI and cloned into pMW172 digested with the same enzymes. Each mAb was tested for binding to the expressed proteins on western blots.

Mixtures of recombinant protein fragments of dystrophin corresponding to exons 6-16, 8-16, 4-16, 7-16, 10-16, and 12-16 were loaded as a strip onto 12% acrylamide gels and separated by SDS-PAGE. Along with the expected dystrophin fragments, degradation products were also present. After electroblotting, monoclonal antibodies were tested on each blot using a miniblotter apparatus as described by Thanh et al. (American Journal of Human Genetics 56:725-731, 1995). The 14 mAbs that were analyzed are shown in Table 1. MANEX1216E does not react with the smallest degradation product and hence recognizes a different epitope from 1216A-D. It is also the only MANEX1216 mAb to recognize native dystrophin in muscle sections. The MANEX7B mAb was selected for further analyses due to strong reactivity to exon 7 and minor reactivity to exon 8.

TABLE 1
Characterization of 14 monoclonal antibodies produced from a
dystrophin fragment encoded by exons 4-16.

| Name | Clone Number | Ig Class | Exon Mapping | IMF | Blot |
|---|---|---|---|---|---|
| MANEX6 | 4H4 | G1 | 6 | Weak | weak |
| MANEX7A | 5D12 | G1 | 7 | weak | weak |
| MANEX7B | 8E11 | G1 | 7 | + | + |
| MANEX7C | 6F7 | G1 | 7 | + | + |
| MANEX1011A | 8A12 | G1 | 10-11 | + | + |
| MANEX1011B | 1C7 | G2a | 10-11 | + | + |
| MANEX1011C | 4F9 | G1 | 10-11 | + | + |
| MANEX1011D | 7G5 | G1 | 10-11 | + | + |
| MANEX1011E | 8H7 | G2a | 10-11 | + | + |
| MANEX1216A | 5A4 | G2a | 12-16 | weak | + |
| MANEX1216B | 6B11 | G2a | 12-16 | weak | + |
| MANEX1216C | 8C8 | G1 | 12-16 | weak | + |
| MANEX1216D | 8D11 | G1 | 12-16 | weak | + |
| MANEX1216E | 2G10 | G1 | 12-16 | + | + |

mAbs were tested for binding to native dystrophin by immunofluorescence microscopy (IMF) of human muscle sections and for binding to denatured dystrophin as determined by separate Western blot (Blot) of human muscle extract. Although two mAbs were “weak” on human muscle blots, they reacted strongly on blots of recombinant protein.

### Detection of Exon 7-Epitope by Western Blotting After Gene Repair

Western blotting of lysed GRMD skeletal muscle was performed according to Arahata et al. (Proceedings of the National Academy of Sciences USA 86:7154-7158, 1989). Ten to 20 frozen sections were collected from untreated triceps muscle, right cranial tibialis (CT) muscle, right long digital extensor (LDE) muscle, left LDE muscle, CT muscle from a normal dog, and left CT muscle. Cryomicrotome sections of 20 μm thickness from samples of various types of canine muscle samples were separately collected and stored at −80° C. until gels were prepared for electrophoresis. Care was taken to be certain that fresh blades were used after positive control samples were sectioned.

Tissue sections were lysed in buffer (1% SDS, 10 mM EDTA, Tris pH 8.0, and 50 mM DTT), boiled for 3 min, then cleared by centrifugation at 14,000 rpm in a microfuge for 5 min. Samples (3-10 11) were loaded onto 3.5-12% laemmli gradient gels with 3% stacking gels and separated in a constant voltage electric field of 60 V per cm for 16 hr. Electroblotting was in transfer buffer (20% methanol, Tris glycine) onto nitrocellulose (Amersham) for 3 hr in a Hoeffer TRANSBLOT electrophoresis chamber. A 1:100 dilution in TBST of the primary antibodies in Table 1 (e.g., exon 7-specific antibody MANEX7B) was incubated for 60 min with the transferred membrane. The membrane was washed extensively and probed with an IMMUNESTAR chemoluminescent kit (goat anti-mouse, BioRad) to detect the MANEX7B mAb bound to the membrane. Kodak XL-R film was exposed for 15 sec, and then processed using a UMAX POWERLOOK II scanner and Photoshop LE computer program. Results were stored on a UMAX Mac-compatible computer.

To investigate whether increases in RT/PCR product containing exon 7 correlated with restoration of normal dystrophin, western blot analyses were performed using the MANEX7B mAb. When samples taken at necropsy were studied using this antibody, restoration of normal sized dystrophin protein containing exon 7 epitope was observed. This is indicative that the treatment with chimera produced a modest level of gene repair detectable at 11 months post injection. While both the left cranial tibialis (CT) muscle, in particular, and the long-digital extensor (LDE) muscle, to a lesser extent, revealed the expected high molecular weight band co-migrating with the normal muscle sample, no significant high molecular weight of dystrophin protein containing exon 7 epitopes was found in the right limb at necropsy. As expected, no high molecular weight protein was found in untreated GRMD muscle samples. Due to limitation of sample size, no samples from the 2, 6 or 9 week timepoints could be included in these analyses. But expression of a normal-sized dystrophin protein containing an epitope encoded by exon 7 was found 11 months after CMV treatment, and provided evidence that modest levels of gene repair of the GRMD mutation had occurred in the left leg.

### Detection of Exon 7-Epitope by Fluorescent Immunohistochemistry After Gene Repair

To determine the pattern of dystrophin distribution in the treated skeletal muscle, an epitope encoded by exon 7 was localized on frozen sections taken at necropsy. Frozen sections were blocked with normal goat serum, incubated with MANEX7B mAb as primary antibody and goat anti-mouse FITC-conjugated secondary antibody (Sigma), and counter-stained with DAPI (15 μg/ml). MANEX7B mAb was localized using an FITC fluorescence bandpass filter while cells were visualized using a triple bandpass filter for DAPI fluorescence. Specificity of the MANEX7B mAb was confirmed by finding that it did not localize to untreated GRMD triceps muscle. In contrast, peripheral staining of a small percentage of fibers was observed in the sections taken from both the right and left cranial tibialis (CT) muscles, while the positive control muscles demonstrated a pattern of normal CT muscle staining of wild-type dystrophin.

As each injected muscle received numerous injections, positive fibers were found in clusters proximal to the injection track and usually were no more than about 2-3 mm from an injection site. Due to limiting sample mass, biopsy samples from the 2 and 9 week were not tested. Interestingly, no exon 7-epitope was found in the right CT muscle at necropsy. But the localization of the exon 7-epitope to the periphery of muscle fibers 11 months after treatment of the left CT muscle further confirms that gene repair of the GRMD mutation has occurred after treatment. The difference between the two treatments was the use of FUGENE™ 6 lipid as a carrier in the left limb. Based on similar results from parallel studies reported previously in the mdx mouse, we suggest that the chimera was more readily introduced into myonuclei using the FuGENE™ 6 lipid carrier, and thus was able to sustain higher levels of long-term expression of functional dystrophin.

### Discussion of Results

In a canine model of Duchenne muscular dystrophy (GRMD), a point mutation within the splice acceptor site of intron 6 leads to deletion of exon 7 from the dystrophin mRNA and the consequent frameshift causes early termination of translation. A hairpin-shaped DNA and RNA chimeric oligonucleobase (i.e., a chimeric mutational vector) was designed to correct the chromosomal mutation to wild-type, possibly by inducing the cell's mismatch repair mechanism. Correction of this point mutation allows appropriate splicing of the dystrophin transcript to include exon 7. Direct injection of the CMV into the skeletal muscle of the cranial tibialis (CT) compartment of a six-week old affected male dog, and subsequent analysis of biopsy and necropsy samples, demonstrated in vivo reversion of the GRMD mutation which was sustained for 11 months. RT/PCR analysis of exons 5-10 demonstrated increasing levels of exon 7 inclusion with time. An exon 7-specific dystrophin antibody confirmed synthesis of normal-sized dystrophin product and positive localization to the sarcolemma. Chromosomal reversion in muscle tissue was confirmed by RFLP/PCR and sequencing the PCR product. This is the first long-term demonstration of reversion of a point mutation in muscle of a live animal using a CMV. In vivo delivery of a CMV and lipid composition provides an alternative to myoblast transplantation or viral gene therapy for the treatment of Duchenne dystrophy and other muscular dystrophies that addresses deficiencies of such methods.

Since the CMV used above actually modifies the mutant gene while maintaining all of the native control elements for dystrophin expression, production of dystrophin from a threshold level of corrected genes would be predicted to permit normalization of dystrophin expression patterns in the skeletal muscle. Expanded studies with multiple animals would also permit force generation analyses to correlate potential strength improvement produced from expression of normalizes dystrophin. Moreover, as the resulting dystrophin gene expression patterns reported here are subclinical, methods to improve the frequency of reversion are under consideration. These improvements would include: 1) higher concentrations of CMV delivered either as a single bolus or in serial administrations, 2) extended delivery via an implantable osmotic pump, 3) addition of carrier molecules such as modified polyethyleneimine (PEI) or ligands targeting skeletal muscle cells, and 4) alternate methods of physical introduction such as electroporation.

Based on a previous report in liver using a chimera to mutate the factor IX gene in rats, higher levels of gene modification were achievable by improving delivery of CMV. A putative clinically-relevant threshold of dystrophin expression to prevent the dystrophic phenotype has been suggested to be 20% of normal levels. Thus, strategies which produce higher levels of reversion may be useful since CMV have little inherent capacity for inducing an immune response. As reported previously for liver, serial administration of CMV in dystrophic muscle might have additive effects and may result in achievement of clinically relevant levels of gene modification which would be measurable by force-generation in this animal model for Duchenne muscular dystrophy.

Furthermore, we believe the GRMD model should also be useful for analyzing the potential of using CMV for restoration of reading frame caused by deletions. The fact that exon 7 is missing from the dystrophin mRNA in dogs with this mutation actually simulates an exon 7 genomic deletion. Thus, a CMV designed to restore reading frame by modifying the coding sequence beginning in exon 8 to match the reading frame from exon 6 would be predicted to produced a protein that wold be Becker-like and may have sufficient function to normalize the muscle in this model.

Murine Model of Muscular Dystrophy

### Design and Synthesis of Chimeric Mutational Vector

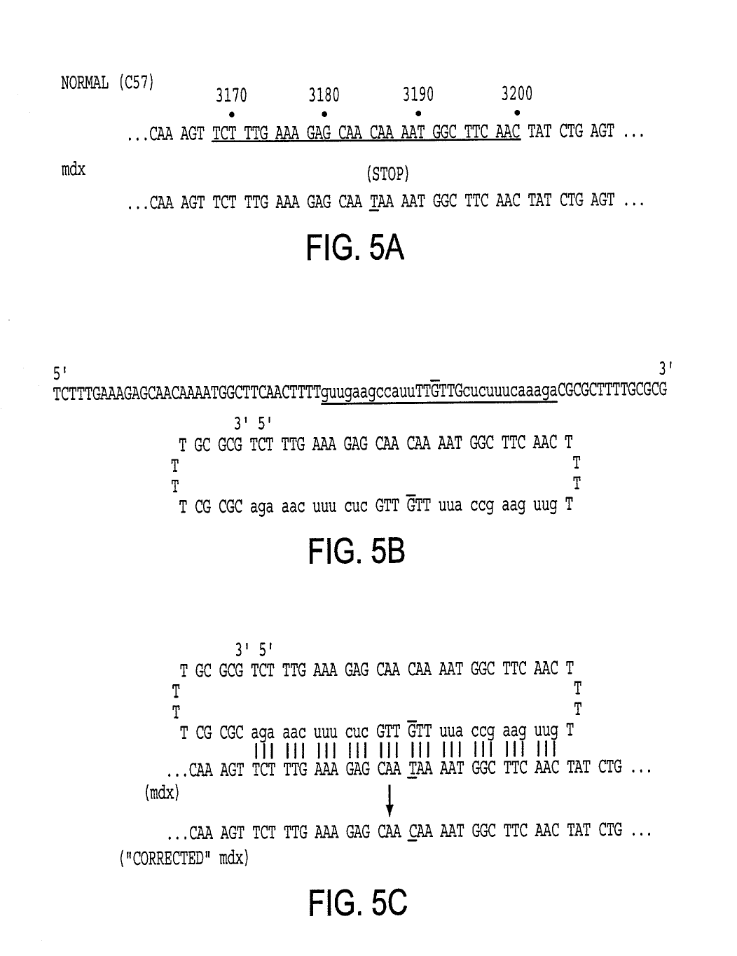

The primary sequence of the CMV, termed MDX1, was designed to correct the point mutation in the mdx dystrophin gene (FIG. 5). Two CMV were used as controls with identical results: one has a sequence homologous to a region of the dog dystrophin gene (a 28-bp region spanning intron 6 and exon 7) and the other was used to the sickle-cell mutation in a globin gene (designated SCI; Cole-Strauss et al., Science 273:1386-1389, 1996). The flanking sequences for both were the same as the flanking sequences in MDX1.

CMV were synthesized as previously described (Sicinski et al., Science 244:1578-1580, 1989). Oligonucleobases were prepared with DNA and 2′-O-methyl RNA phosphoramidite nucleoside monomers on a Perseptive Biosystems Expedite Nucleic Acid Synthesizer, purified by HPLC and quantified by UV absorbance. The Cy3-MDX1 CMV were purified using ABI OPC reverse phase purification cartridges and ethanol precipitated twice. More than 95% of the purified oligonucleobases were determined to be of full length.

### Direct Injection of CMV for Gene Repair

Mice of the mdx strain (C57BL/10ScSn-mdx) were obtained from Jackson Lab (Bar Harbor, Me.) and were handled in accordance with guidelines of the Administrative Panel on Laboratory Animal Care of Stanford University. Mice were anesthetized with a ketamine/xylazine cocktail (doses: 125 mg/kg ketamine; 25 mg/kg xylazine). For each injection, the skin over the tibialis anterior muscle was shaved, sterilized, and incised. CMV was dissolved in PBS at a concentration of 4 mg/ml, and the solution was drawn up into a 10 μl Hamilton syringe with a 30 gauge needle. The needle was inserted along the rostro-caudal axis of the muscle into the center of the muscle belly, and 20 μg of the CMV solution was injected in a volume of 5 μl. After the injection, the skin was sutured closed.

### Histologic and Fluorescent Immunohistochemical Analyses

Mice were sacrificed at different times after CMV injection, and the tibialis anterior muscles were dissected. The muscles were embedded in OCT mounting compound (Miles), frozen in isopentane cooled in liquid nitrogen, and stored at −80° C. Frozen sections were collected on gelatin-coated slides and stored at −20° C. Serial cross-sections (7 μm thick) were collected along the entire length of the muscle at intervals of 200-300 μm.

Alternatively, for analysis of Cy3 fluorescence after injection of Cy3-MDX1 CMV, muscle sections were warmed to room temperature, hydrated in PBS for 5 min, and cover-slipped using an aqueous mounting medium. Sections were examined using a Zeiss Axioskop fluorescent microscope.

For dystrophin immunohistochemical staining, an antibody directed against the rod domain of the dystrophin protein (MANDYS-8; Sigma) was used at a dilution of 1:400. Specific antibody binding was detected with an Alexa-coupled, goat-anti-mouse secondary antibody (Molecular Probes) at a dilution of 1:1000. Controls for specific staining included sections treated with no primary antibody. The number of dystrophin-positive fibers in a given muscle was determined in the serial section containing the greatest number of fibers. To test for revertant fibers, an antibody directed against the protein product encoded by exon 26 of the dystrophin gene (MANDYS-18; a gift from Dr. Glenn Morris) was used at a dilution of 1:3 in place of the MANDYS-8 antibody.

For routine histological analysis, sections adjacent to those processed for fluorescence microscopy were stained with hematoxylin and eosin (H&E). The needle track was easily identified in H&E-stained sections both by the characteristic changes in muscle architecture created by the needle injury and by the reproducible location in the muscle. Furthermore, in muscles injected with Cy3-MDX1, the distribution of the fluorochrome corresponded exactly with the location of the needle track identified in H&E-stained adjacent sections.

### Immunoprecipitation and Immunoblot Analyses

For immunoblot analysis, muscles were dissected and homogenized in RIPA buffer consisting of 150 mM NaCl, 50 mM Tris-HCl, 5 mM EDTA, 5 mM EGTA, 0.5% deoxycholate, 1% NP40, 20 μg/ml leupeptin, 20 μg/ml aprotinin, 100 μg/ml PMSF, and 50 mM DTT. For each sample, the protein concentration was determined using the Bio-Rad protein assay. When dystrophin was immunoprecipitated prior to electrophoresis, equal amounts of protein (6 mg) from precleared extract were immunoprecipitated using the MANDYS-8 anti-dystrophin antibody (1:100) for 3 hr on ice, followed by protein-G-agarose for 1 hour. Samples were run on 5% SDS-polyacrylamide gels, transferred to 0.45 μm nitrocellulose membranes (Schleicher and Schuell), and probed with mouse monoclonal antibodies to dystrophin (MANDYS-8, 1:400 dilution, or MANDYS-18, 1:100 dilution) followed by a horseradish peroxidase-coupled sheep-anti-mouse secondary antibody. Specific antibody binding was detected by an enhanced chemiluminescence system (Amersham).

### Distribution of Injected CMV

In order to assess first the uptake and distribution of the CMV after injection, fluorochrome-coupled MDX1 was injected into the tibialis anterior muscles of mdx mice. The distribution of the fluorescent label was examined in muscle sections at different times after injection and was very characteristic. Labeled fibers were seen in two contiguous areas—a linear pattern defining the track of the needle and a cluster at the end of the needle track at the actual injection site. This pattern was clearly discernible 4 hr after injection and persisted with little apparent change over the next 24 hr. By 48 hr after injection, the intensity of the fluorescent signal was greatly diminished, and it was barely detectable 72 hr after injection. Presumably, this decline in signal represents the metabolism of the CMV and provides some evidence of the stability of these molecules in the cell.

### Dystrophin Expression in MDX1-Injected Muscles

To test the efficacy of MDX1 to effect gene repair in mdx mouse muscle, tissue sections were examined for dystrophin expression two weeks after MDX1 injections. Expression was seen only along the needle track and at the injection site. Dystrophin immunohistochemical staining around the injection site in two muscles injected with MDX1 was also examined. In each muscle, dystrophin-positive fibers were detected in a pattern similar to the pattern of fluorescent label seen with the fluorochrome-labeled CMV, either along a linear track or in a small cluster. When control CMV were injected, no dystrophin-positive fibers were detected in the vicinity of the injection site.

In order to obtain a quantitative measure of the efficacy of this procedure, the number of dystrophin positive fibers was counted two weeks after a single MDX1 injection in a series of muscles. The number of dystrophin-positive fibers ranged from a low of nine to a high of 32 in these muscles. These numbers represent a range of about 10-20% of the number of fibers brightly stained by fluorescent CMV 24 hr after injection. Thus, only a subset of fibers that took up the CMV produced sufficient dystrophin to be detected by immunohistochemical staining.

### Detection of Revertant Fibers

In mdx mouse muscle as well as in human muscle from patients with DMD, there is an increase in the appearance of dystrophin-positive fibers, so-called ‘revertant’ fibers, with age (Hoffman et al., Journal of Neurological Sciences 99:9-25, 1990). For mdx muscle, the molecular basis of this reversion has been postulated to be spontaneous, somatic mutations resulting in either in-frame deletions around and including exon 23 (which contains the point mutation), or alternative splicing reactions which would produce transcripts that excluded exon 23. This hypothesis is supported by analysis of revertant fibers with exon-specific antibodies to dystrophin and by nested PCR analysis of transcripts in mdx and DMD muscle (Wilton et al., Muscle and Nerve 20:728-734, 1997; Thanh et al., American Journal of Human Genetics 56:725-731, 1995).

The negative results with the control CMV argue against any non-specific (i.e., sequence-independent) effect of the experimental procedures leading to an increase in the number of revertant fibers as an explanation for the dystrophin-positive fibers seen after MDX1 injection. Still, to rule out this possibility with greater certainty, antibodies directed against the protein products of exons that are rarely, if ever, expressed in revertant fibers (generally, exons 20-30) were used (Wilton et al., Muscle and Nerve 20:728-734, 1997; Lu & Partridge, Journal of Histochemistry and Cytochemistry 46:977-983, 1998).

A monoclonal antibody directed against exon 26 stained the same fibers as those detected with the antibody directed against a distant region of the dystrophin protein, providing further evidence that dystrophin expression in MDXI -injected muscles was not due to an increased generation of revertant fibers. When the exon 26-specific antibody was used to stain the rare dystrophin-positive fibers away from the site of injection, the staining was negative as would be expected for a revertant fiber (Wilton et al., Muscle and Nerve 20:728-734, 1997; Lu & Partridge, Journal of Histochemistry and Cytochemistry 46:977-983, 1998).

As a further demonstration that the dystrophin immunoreactivity found in MDX1-injected muscle represented a correction of the point mutation and thus the expression of full-length dystrophin, the muscles were examined for dystrophin expression by immunoblot analysis. Because of the low number of dystrophin-positive fibers seen in muscle sections, dystrophin expression was undetectable by standard Western blot analysis. This was not surprising since the percentage of dystrophin-positive fibers generated from MDX1 injections in any given muscle was, at best, approximately 1-2% of the total number of fibers. Therefore, an anti-dystrophin antibody was used to immunoprecipitate any dystrophin that might be present, and the immunoprecipitate was then subjected to immunoblot analysis. Using this approach, a single band was detected at a molecular weight corresponding to full-length dystrophin (427 kd) in MDX1-injected muscles. In muscles injected with control CMV, no such band was detected. That MDX1 is inducing single-base exchange, thus correcting the mdx mutation, is supported by the finding of full-length dystrophin by immunoblot analysis. The generation of revertants by somatic deletions or alternative splicing would be expected to produce truncated forms of the protein.

CMV are taken up into mature myofibers as evidenced by the appearance of fluorescent label in myofibers within 4 hr of injection of fluorescently labeled compounds. Expression of dystrophin in mature fibers within two weeks of injection of MDX1 chimeric mutational vector suggests that CMV-induced gene correction may occur in post-mitotic cells. However, it is also possible that the gene correction event could have occurred in proliferating myoblasts which subsequently fused with the mature fibers. Experiments are ongoing to test this possibility by injuring muscle to stimulate myoblast proliferation prior to CMV injection.

Results confirming the above are published as Rando et al., Proceedings of the National Academy of Sciences USA 97:5363-5368, 2000; Bartlett et al., Nature Biotechnology, in the press, June 2000); and Alexeev et al., Nature Biotechnology 18:43-47, 2000.

The foregoing description represents only certain embodiments and technical features of the invention. It should be understood that persons of ordinary skill in the art could make various modifications and substitutions without departing from the spirit of this invention (e.g., modification of the CMV sequence to correct other mutations in the dystrophin gene; substitution of other lipids for the FuGENETm 6 lipid; modification of the transfection method and substitution of transfection agents). In particular, all combinations of the embodiments and technical features described herein are also considered to be within the scope of the invention.

The appended claims describe what are considered patentable aspects of the invention. But although the claims are read in light of this specification, any particular embodiment or technical feature described in this specification would not limit those claims unless it was also explicitly recited therein. Therefore, legal protection for this invention can only be determined by reference to the issued claims and equivalents thereof with the proviso that the prior art is excluded from coverage.

All patents, patent applications, books, and other references cited herein are indicative of the level of skill in the art and are incorporated by reference where they are cited.

We claim:

1. A composition for the correction of a mutated dystrophin gene comprising an oligonucleobase having both ribo-type and deoxyribo-type nucleobases, which oligonucleobase comprises:
a) a first and a second homologous region that are each at least eight nucleobases in length and together at least 20 and not more than 60 nucleobases in length, in which the homologous regions are, respectively, homologous to a first fragment and a second fragment of an exon of human dystrophin or of such exon and its 5′ or 3′ flanking intron, in which each homologous region comprises at least three nucleobases of hybrid-duplex, and
b) a heterologous region that is disposed between the first and second homologous region;
wherein the composition is effective in correcting the mutated dystrophin gene in at least some muscle cells by in vivo administration.

2. The composition of claim 1 further comprising a lipid effective in introducing the oligonucleobase into at least some muscle cells by in vivo administration.

3. The composition of claim 2 which consists essentially of the oligonucleobase and FUGENE™ 6 lipid.

4. The composition of claim 1, wherein the oligonucleobase is linked by a covalent linker to a ligand that targets the oligonucleobase to a muscle cell.

5. A method of correcting a mutation in the dystrophin gene of muscle tissue in an affected subject, which comprises:
providing a composition comprising an oligonucleobase having both ribo-type and deoxyribo-type nucleobases, which oligonucleobase comprises:
a) a first and a second homologous region that are each at least eight nucleobases in length and together at least 20 and not more than 60 nucleobases in length, in which the homologous regions are, respectively, homologous to a first fragment and a second fragment of the dystrophin gene of the subject, which fragments are each adjacent to the point mutation, and in which each homologous region comprises at least three nucleobases of hybrid-duplex, and
b) a heterologous region that is disposed between the first and second homologous region; and
administering to the subject an amount of the composition that is effective in vivo to correct the mutation in at least some muscle cells of the subject.

6. The method of claim 5, wherein the composition further comprises a lipid effective in introducing the oligonucleobase into at least some muscle cells by in vivo administration.

7. The method of claim 6, wherein the composition consists essentially of the oligonucleobase and FUGENE™ 6 lipid.

8. The method of claim 5, wherein the first and second fragment are fragments of an exon of the dystrophin gene or of such exon and the 3′ or 5′ flanking intron of the exon.

9. The method of claim 5, wherein the composition is administered to the subject by intra-muscular injection.

10. The method of claim 5, wherein the oligonucleobase is linked by a covalent linker to a ligand that targets the oligonucleobase to a muscle cell.

11. The method of claim 5, wherein the subject is canine or murine.

12. The method of claim 5, wherein the subject is a human and the mutation is corrected in somatic cells without effecting the germline.

13. A method of correcting an inherited or acquired mutation in affected cells of a subject, which comprises:
providing a composition comprising an oligonucleobase having both ribo-type and deoxyribo-type nucleobases, which oligonucleobase comprises:
a) a first and a second homologous region that are each at least eight nucleobases in length and together at least 20 and not more than 60 nucleobases in length, in which the homologous regions are, respectively, homologous to a first fragment and a second fragment of a gene with the inherited or acquired mutation, and in which each homologous region comprises at least three nucleobases of hybrid-duplex, and
b) a heterologous region that is disposed between the first and second homologous region; and
administering to the subject an amount of the composition that is effective in vivo to correct the mutation in at least some cells of the subject's affected tissue.

14. The method of claim 13, wherein the composition further comprises a lipid effective in introducing the oligonucleobase into at least some muscle cells by in vivo administration.

15. The method of claim 14, wherein the composition consists essentially of the oligonucleobase and FUGENE™ 6 lipid.

16. The method of claim 13, wherein the first and second fragment are fragments of an exon of the dystrophin gene or of such exon and the 3′ or 5′ flanking intron of the exon.

17. The method of claim 13, wherein the composition is administered to the subject by intramuscular injection.

18. The method of claim 13, wherein the oligonucleobase is linked by a covalent linker to a ligand that targets the oligonucleobase to a muscle cell.

19. The method of claim 13, wherein the subject is canine or murine.

20. The method of claim 13, wherein the subject is a human and the mutation is corrected in somatic cells without effecting the germline.
